SEQUENCE LISTING (103 sequences)

SEQ ID NO: 1 - protein, 1273 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1260 aa, Severe acute respiratory syndrome-related coronavirus
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 1259 aa, synthetic construct
QCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPQQAQSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 1258 aa, synthetic construct
QCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 1260 aa, synthetic construct
QCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 1257 aa, synthetic construct
QCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHQQAQSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - protein, 1258 aa, synthetic construct
QCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGITAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - protein, 1258 aa, synthetic construct
QCVNLTTRTQLPPAYTNSFTRGVYYPDKVFRSSVLHSTQDLFLPFFSNVTWFHVIHVSGTNGTKRFDNPVLPFNDGVYFASIEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINIVRDLPQGFSALEPLVDLPIGINITRFQTLLALHRSYLPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - protein, 1257 aa, synthetic construct
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLGVSNHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 1261 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLGVTSNHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMKSEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAHEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 14 - protein, 942 aa, synthetic construct
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 16 - protein, 1260 aa, Severe acute respiratory syndrome-related coronavirus; identical to SEQ ID NO: 2

SEQ ID NO: 17 - protein, 1250 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPQQAQSVALGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 18 - protein, 1250 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPQQAQSVASQSIIAYTMSLGAENSVAYSNNSIAIEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 19 - protein, 1250 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 20 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 21 - protein, 1266 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTHHHHHH

SEQ ID NO: 22 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 23 - protein, 1257 aa, synthetic construct; identical to SEQ ID NO: 7

SEQ ID NO: 24 - protein, 1260 aa, synthetic construct
QCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPQQAQSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 25 - protein, 1259 aa, synthetic construct; identical to SEQ ID NO: 4

SEQ ID NO: 26 - protein, 1258 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 27 - protein, 1255 aa, synthetic construct
QCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHGGSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 28 - protein, 1257 aa, synthetic construct
QCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGGSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 29 - protein, 1256 aa, synthetic construct
QCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPQQAQSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 30 - protein, 1254 aa, synthetic construct
QCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGGSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 31 - protein, 1260 aa, synthetic construct; identical to SEQ ID NO: 6

SEQ ID NO: 32 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSCMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQPYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 33 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGGSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPQQAQSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 34 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGGSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGNTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPQQAQSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 35 - protein, 1258 aa, synthetic construct; identical to SEQ ID NO: 9

SEQ ID NO: 36 - protein, 1257 aa, synthetic construct; identical to SEQ ID NO: 10

SEQ ID NO: 37 - protein, 1258 aa, synthetic construct; identical to SEQ ID NO: 5

SEQ ID NO: 38 - protein, 1260 aa, synthetic construct; identical to SEQ ID NO: 13

SEQ ID NO: 39 - protein, 1258 aa, synthetic construct; identical to SEQ ID NO: 8

SEQ ID NO: 40 - protein, 1260 aa, synthetic construct; identical to SEQ ID NO: 11

SEQ ID NO: 41 - protein, 1261 aa, synthetic construct; identical to SEQ ID NO: 12

SEQ ID NO: 42 - protein, 1257 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 43 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 44 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNNLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTVSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 45 - protein, 1257 aa, synthetic construct
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 46 - protein, 1257 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVRQIAPGQTGNIADYNYKLPDDFTGCVIAWNSNKLDSKVGGNYNYQYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENLVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 47 - protein, 1257 aa, synthetic construct; identical to SEQ ID NO: 10

SEQ ID NO: 48 - protein, 1257 aa, synthetic construct; identical to SEQ ID NO: 42

SEQ ID NO: 49 - protein, 1255 aa, synthetic construct; identical to SEQ ID NO: 43

SEQ ID NO: 50 - protein, 1255 aa, synthetic construct; identical to SEQ ID NO: 44

SEQ ID NO: 51 - protein, 1257 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYQYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENLVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 52 - protein, 1257 aa, synthetic construct; identical to SEQ ID NO: 46

SEQ ID NO: 53 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNSSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 54 - protein, 1257 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHENNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGFNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 55 - protein, 1257 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHENNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYRYRLFRKSKLKPFERDISTEIYQAGNKPCNGVAGSNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNISNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLINLQELGKYEQYIKWPWYIWLGFIAGLIAIVMVTIMLCCMTSCCSCLKGCCSCGSCCKFDEDDSEPVLKGVKLHYT

SEQ ID NO: 56 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 57 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 58 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 59 - protein, 1254 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGSNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 60 - protein, 1256 aa, synthetic construct
QCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGSNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 61 - protein, 1254 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 62 - protein, 1257 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHXNNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRTRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGFNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 63 - protein, 1270 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 64 - protein, 1270 aa, synthetic construct
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHENNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGFNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 65 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNSSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 66 - protein, 1270 aa, synthetic construct
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHENNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYRYRLFRKSKLKPFERDISTEIYQAGNKPCNGVAGSNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNISNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLINLQELGKYEQYIKWPWYIWLGFIAGLIAIVMVTIMLCCMTSCCSCLKGCCSCGSCCKFDEDDSEPVLKGVKLHYT

SEQ ID NO: 67 - protein, 4 aa, Severe acute respiratory syndrome-related coronavirus
RRAR

SEQ ID NO: 68 - protein, 4 aa, synthetic construct
QQAQ

SEQ ID NO: 69 - protein, 4 aa, synthetic construct
QRAR

SEQ ID NO: 70 - protein, 4 aa, synthetic construct
RQAR

SEQ ID NO: 71 - protein, 4 aa, synthetic construct
RRAQ

SEQ ID NO: 72 - protein, 4 aa, synthetic construct
QQAR

SEQ ID NO: 73 - protein, 4 aa, synthetic construct
RQAQ

SEQ ID NO: 74 - protein, 4 aa, synthetic construct
QRAQ

SEQ ID NO: 75 - protein, 4 aa, synthetic construct
NNAN

SEQ ID NO: 76 - protein, 4 aa, synthetic construct
NRAR

SEQ ID NO: 77 - protein, 4 aa, synthetic construct
RNAR

SEQ ID NO: 78 - protein, 4 aa, synthetic construct
RRAN

SEQ ID NO: 79 - protein, 4 aa, synthetic construct
NNAR

SEQ ID NO: 80 - protein, 4 aa, synthetic construct
RNAN

SEQ ID NO: 81 - protein, 4 aa, synthetic construct
NRAN

SEQ ID NO: 82 - protein, 4 aa, synthetic construct
AAAA

SEQ ID NO: 83 - protein, 4 aa, synthetic construct
ARAR

SEQ ID NO: 84 - protein, 4 aa, synthetic construct
RAAR

SEQ ID NO: 85 - protein, 4 aa, synthetic construct
RRAA

SEQ ID NO: 86 - protein, 4 aa, synthetic construct
AAAR

SEQ ID NO: 87 - protein, 4 aa, synthetic construct
RAAA

SEQ ID NO: 88 - protein, 4 aa, synthetic construct
ARAA

SEQ ID NO: 89 - protein, 4 aa, synthetic construct
GGAG

SEQ ID NO: 90 - protein, 4 aa, synthetic construct
GRAR

SEQ ID NO: 91 - protein, 4 aa, synthetic construct
RGAR

SEQ ID NO: 92 - protein, 4 aa, synthetic construct
RRAG

SEQ ID NO: 93 - protein, 4 aa, synthetic construct
GGAR

SEQ ID NO: 94 - protein, 4 aa, synthetic construct
RGAG

SEQ ID NO: 95 - protein, 4 aa, synthetic construct
GRAG

SEQ ID NO: 96 - protein, 4 aa, synthetic construct
GSAS

SEQ ID NO: 97 - protein, 4 aa, synthetic construct
GSGA

SEQ ID NO: 98 - protein, 805 aa, Homo sapiens [sequence omitted]

SEQ ID NO: 99 - protein, 788 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSF

SEQ ID NO: 100 - protein, 10 aa, synthetic construct
  VARIANT 6..10: note = individual H residues may be present or absent
HHHHHHHHHH

SEQ ID NO: 101 - protein, 6 aa, synthetic construct
HHHHHH

SEQ ID NO: 102 - protein, 7 aa, synthetic construct
HHHHHHH

SEQ ID NO: 103 - protein, 8 aa, synthetic construct
HHHHHHHH

(19) United States
(12) Patent Application Publication
(10) Pub. No.: US 2026/0309709 A1
(43) Pub. Date: Oct. 8, 2026

(54) ACE2 INHIBITION ASSAY FOR EVALUATION OF VACCINE IMMUNOGENICITY

(71) Applicant: NOVAVAX, INC., Gaithersburg, MD (US)

(72) Inventors: Joyce S. PLESTED, Gaithersburg, MD (US); Raj KALKERI, Gaithersburg, MD (US)

(21) Appl. No.: 18/998,471

(22) PCT Filed: Jul. 26, 2023
(86) PCT No.: PCT/US2023/071044

§ 371 Date: Jan. 25, 2025

Related U.S. Application Data
(60) Provisional application No. 63/392,390, filed on Jul. 26, 2022.
(60) Provisional application No. 63/405,653, filed on Sep. 12, 2022.
(60) Provisional application No. 63/428,991, filed on Nov. 30, 2022.

(51) Int. Cl. G01N 33/569

(52) U.S. Cl. CPC G01N 33/56983; G01N 2333/165; G01N 2333/948; G01N 2469/20

(57) ABSTRACT

An improved assay to measure immunogenicity of vaccines while overcoming some of the limitations of current biomarkers of immunogenicity is necessary. Disclosed herein is an assay for measuring inhibition of binding between SARS-CoV-2 Spike (S) glycoproteins and hACE2. Also provided herein are methods of using the assay to evaluate the efficacy of COVID-19 vaccines.

### CROSS-REFERENCE TO RELATED APPLICATIONS

This application claims priority to U.S. Provisional Application No. 63/392,390, filed on Jul. 26, 2022; U.S. Provisional Application No. 63/405,653, filed on Sep. 12, 2022; and U.S. Provisional Application No. 63/428,991, filed Nov. 30, 2022. Each of these applications is hereby incorporated by reference in its entirety for all purposes.

### REFERENCE TO AN ELECTRONIC SEQUENCE LISTING

The contents of the electronic sequence listing (NOVV-102_03WO_SeqList_ST26.xml; Size: 174,555 bytes; and Date of Creation: Jul. 26, 2023) are herein incorporated by reference in its entirety.

### FIELD

The present disclosure is generally related to methods for identifying if a biological sample (e.g., serum, blood, plasma) contains antibodies that inhibit the interaction between human angiotensin converting enzyme 2 (hACE2) and SARS-CoV-2 S glycoproteins.

### BACKGROUND

Biomarkers of immunogenicity, which may eventually be proven to be correlates of protection, are critical for assessment of vaccines. Some of the current biomarkers used to assess vaccine immunogenicity of COVID-19 vaccines are anti-spike (S) or anti-receptor binding domain (RBD) immunoglobulin G (IgG) antibodies, neutralizing antibody responses, and levels of activated T cells. Each of these markers has limitations. Neither anti-S nor anti-RBD IgG shows neutralization function of the antibodies. Neutralizing antibodies measured by microneutralization assays may block infection, but may not be specific to a certain target or be homogenous. Higher counts of CD4+/CD8+ T cells specific for SARS-CoV-2 epitopes may be associated with reduced severity of infection but are more difficult to measure, and their role in COVID-19 vaccine immunogenicity is still unclear. Additionally, emerging variants of SARS-CoV-2 such as Omicron have shown differences in reliability as correlates of protection. An improved assay to measure immunogenicity of vaccines while overcoming the limitations of current biomarkers is necessary.

### SUMMARY OF THE INVENTION

Provided herein is a new biomarker of COVID-19 vaccine immunogenicity, inhibition of SARS-CoV-2 spike (S) protein binding to the hACE2 receptor. This assay provides a rapid, high-throughput option to evaluate vaccine immunogenicity. Along with other clinical biomarkers, it can provide valuable insights into immune evasion and correlates of protection and enable vaccine development against emerging COVID-19 variants.

Also provided herein is an assay for measuring inhibition of hACE2 binding as a biomarker of COVID-19 vaccine immunogenicity.

Provided herein is a method for determining if a biological sample contains antibodies that inhibit binding of a SARS-CoV-2 S glycoprotein to human angiotensin-converting enzyme 2 (hACE2), comprising: (a) measuring binding of a SARS-CoV-2 S glycoprotein to hACE2 after exposure to a biological sample by: (i) exposing a surface coated with the SARS-CoV-2 S glycoprotein to the biological sample; (ii) exposing the surface to hACE2; and (iii) detecting hACE2 that is bound to the surface; (b) measuring binding of a SARS-CoV-2 S glycoprotein to hACE2 in the absence of the biological sample of (a) by: (i) exposing a surface coated with the SARS-CoV-2 S glycoprotein to hACE2; and (ii) detecting hACE2 that is bound to the surface; and (c) comparing binding of the SARS-CoV-2 S glycoprotein to hACE2 in (a) and (b); wherein a biological sample contains antibodies that inhibit binding of the SARS-CoV-2 S glycoprotein to hACE2 if the amount of bound hACE2 in (a) is less than the amount of bound hACE2 in (b). In embodiments, the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of SEQ ID NO: 1, SEQ ID NO: 2, or SEQ ID NO: 3. In embodiments, the SARS-CoV-2 S glycoprotein has an inactive furin cleavage site. In embodiments, the inactive furin cleavage site has the amino acid sequence of QQAQ (SEQ ID NO: 68). In embodiments, amino acids 973 and 974 of the SARS-CoV-2 S glycoprotein are proline, as compared to a wild-type SARS-CoV-2 S glycoprotein having the amino acid sequence of SEQ ID NO: 2. In embodiments, the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 3, 5-13, 15, 17-19, 21, and 23-66. In embodiments, the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 3-13, 20, and 22-66. In embodiments, the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 3, 5-13, and 23-66. In embodiments, the SARS-CoV-2 S glycoprotein is from a SARS-CoV-2 virus or a variant of SARS-CoV-2. In embodiments, the variant of SARS-CoV-2 is a B.1.1.7 SARS-CoV-2 strain; a B.1.351 SARS-CoV-2 strain; a P.1 SARS-CoV-2 strain; a Cal.20C SARS-CoV-2 strain; a B.1.617.2 SARS-CoV-2 strain; a B.1.525 SARS-CoV-2 strain; a B.1.526 SARS-CoV-2 strain; a B.1.617.1 SARS-CoV-2 strain; a C.37 SARS-CoV-2 strain; a B.1.621 SARS-CoV-2 strain; or a B.1.1.529 SARS-CoV-2 strain. In embodiments, the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a SARS-CoV-2 S glycoprotein from a SARS-CoV-2 S omicron variant selected from the group consisting of: BA.1, BA.2.12.1, BA.2, BA.3, BA.4, BA.5, XBB.1.5, XBB.2.3, and XBB.1.16. In embodiments, the hACE2 is attached to a tag. In embodiments, the tag is a His tag. In embodiments, the biological sample is serum, plasma, or blood from a patient that has previously had COVID-19. In embodiments, the biological sample is serum, plasma, or blood from a patient that has been administered an immunogenic composition against a SARS-CoV-2 virus or a variant thereof. In embodiments, the SARS-CoV-2 S glycoprotein includes the transmembrane domain.

### BRIEF DESCRIPTION OF THE DRAWINGS

FIG. 1 shows a schematic of the assay described in Example 1.

FIG. 2 shows formulas used to calculate the percent geometric coefficient of variation (% GCV) in Example 1.

FIG. 3 shows the formula used to calculate the total % GCV in Example 1.

FIG. 4 shows the formula used to calculate the % reduction in Example 1.

FIG. 5 shows the formula used to calculate the % relative bias in Example 1.

FIG. 6 shows the formula used to calculate the percent recovery in Example 1.

FIG. 7 shows linearity of the hACE2 Binding Inhibition Assay of Example 1.

FIG. 8 shows the stability of the samples of Example 1.

FIGS. 9A-9D show that the assay performs similarly when a SARS-CoV-2 S glycoprotein from the prototype strain (SARS-CoV-2 S glycoprotein having the amino acid sequence of SEQ ID NO: 2) (FIG. 9A); a SARS-CoV-2 S glycoprotein from the SARS-CoV-2 Delta strain (FIG. 9B); a SARS-CoV-2 S glycoprotein from the Omicron BA.1 strain (FIG. 9C); and a SARS-CoV-2 S glycoprotein from the Omicron BA.5 strain are used (FIG. 9D).

FIG. 10A shows that the results of the assay of Example 1 (hACE2 binding inhibition) is significantly correlated with anti-SARS-CoV-2 S IgG titers for the prototype strain (SARS-CoV-2 S glycoprotein having amino acid sequence of SEQ ID NO: 2).

FIG. 10B shows that the results of the assay of Example 1 (hACE2 binding inhibition) are significantly correlated with anti-SARS-CoV-2 S IgG titers for a SARS-CoV-2 Omicron BA.1 Strain.

FIG. 10C shows that the results of the assay of Example 1 are significantly correlated with neutralizing antibody titers for the prototype strain (SARS-CoV-2 S glycoprotein having amino acid sequence of SEQ ID NO: 2).

FIG. 10D shows that the results of the assay of Example 1 are significantly correlated with neutralizing antibody titers for a SARS-CoV-2 Omicron BA.1 Strain.

FIG. 10E shows that the results of the assay of Example 1 (hACE2 binding inhibition) is significantly correlated with anti-SARS-CoV-2 S IgG titers for the prototype strain (SARS-CoV-2 S glycoprotein having amino acid sequence of SEQ ID NO: 2) without the data points at the LOD.

FIG. 10F shows that the results of the assay of Example 1 (hACE2 binding inhibition) are significantly correlated with anti-SARS-CoV-2 S IgG titers for a SARS-CoV-2 Omicron BA.1 Strain without the data points at the LOD.

FIG. 10G shows that the results of the assay of Example 1 (hACE2 binding inhibition) are significantly correlated with anti-SARS-CoV-2 S IgG titers for a SARS-CoV-2 Omicron BA.5 Strain.

FIG. 10H shows that there is a strong correlation between hACE2 binding inhibition titers and neutralizing antibodies.

### DETAILED DESCRIPTION OF THE INVENTION

### Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “adjuvant” refers to a compound that, when used in combination with an immunogen, augments or otherwise alters or modifies the immune response induced against the immunogen. Modification of the immune response may include intensification or broadening the specificity of either or both antibody and cellular immune responses.

As used herein, the term “about” or “approximately” when preceding a numerical value indicates the value plus or minus a range of 10%. For example, “about 100” encompasses 90 and 110.

As used herein, the terms “immunogen,” “antigen,” and “epitope” refer to substances such as proteins, including glycoproteins, and peptides that are capable of eliciting an immune response.

As used herein, an “immunogenic composition” is a composition that comprises an antigen where administration of the composition to a subject results in the development in the subject of a humoral and/or a cellular immune response to the antigen.

The terms “treat,” “treatment,” and “treating,” as used herein, refer to an approach for obtaining beneficial or desired results, for example, clinical results. For the purposes of this disclosure, beneficial or desired results may include inhibiting or suppressing the initiation or progression of an infection or a disease; ameliorating, or reducing the development of, symptoms of an infection or disease; or a combination thereof.

“Prevention,” as used herein, is used interchangeably with “prophylaxis” and can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein an “effective dose” or “effective amount” refers to an amount of an immunogen sufficient to induce an immune response that reduces at least one symptom of pathogen infection. An effective dose or effective amount may be determined e.g., by measuring amounts of neutralizing secretory and/or serum antibodies, e.g., by plaque neutralization, complement fixation, enzyme-linked immunosorbent (ELISA), or microneutralization assay.

As used herein, the term “vaccine” refers to an immunogenic composition, such as an immunogen derived from a pathogen, which is used to induce an immune response against the pathogen that provides protective immunity (e.g., immunity that protects a subject against infection with the pathogen and/or reduces the severity of the disease or condition caused by infection with the pathogen). The protective immune response may include formation of antibodies and/or a cell-mediated response. Depending on context, the term “vaccine” may also refer to a suspension or solution of an immunogen that is administered to a subject to produce protective immunity.

As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular aspects, the subject is up to 4 months old, or up to 6 months old. In aspects, the adults are seniors about 65 years or older, or about 60 years or older. In aspects, the subject is a pregnant woman or a woman intending to become pregnant. In other aspects, subject is not a human; for example a non-human primate; for example, a baboon, a chimpanzee, a gorilla, or a macaque. In certain aspects, the subject may be a pet, such as a dog or cat.

As used herein, the term “pharmaceutically acceptable” means being approved by a regulatory agency of a U.S. Federal or a state government or listed in the U.S. Pharmacopeia, European Pharmacopeia or other generally recognized pharmacopeia for use in mammals, and more particularly in humans. These compositions can be useful as a vaccine and/or antigenic compositions for inducing a protective immune response in a vertebrate.

As used herein, the term “modification” as it refers to a CoV S polypeptide refers to mutation, deletion, or addition of one amino acid of the CoV S polypeptide. The location of a modification within a CoV S polypeptide can be determined based on aligning the sequence of the polypeptide to SEQ ID NO: 1 (CoV S polypeptide containing signal peptide) or SEQ ID NO: 2 (mature CoV S polypeptide lacking a signal peptide).

The term SARS-CoV-2 “variant”, used interchangeably herein with a “heterogeneous SARS-CoV-2 strain,” refers to a SARS-CoV-2 virus comprising a CoV S polypeptide having one or more modifications as compared to a SARS-CoV S polypeptide having the amino acid sequence of SEQ ID NO: 2. For example, a SARS-CoV-2 variant may have at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, or at least about 35 modifications, as compared to a CoV S polypeptide having the amino acid sequence of SEQ ID NO: 2. For example, a SARS-CoV-2 variant may have at least one and up to 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25, up to 26, up to 27, up to 28, up to 29, up to 30, up to 31, up to 32, up to 33, up to 34, up to 35 modifications, up to 40 modifications, up to 45 modifications, up to 50 modifications, up to 55 modifications, up to 60 modifications, up to 65 modifications, up to 70 modifications, up to 75 modifications, up to 80 modifications, up to 85 modifications, up to 90 modifications, up to 95 modifications, or up to 100 modifications as compared to a CoV S polypeptide having the amino acid sequence of SEQ ID NO: 2. In aspects, a SARS-CoV-2 variant may have between about 2 and about 35 modifications, between about 5 and about 10 modifications, between about 5 and about 20 modifications, between about 10 and about 20 modifications, between about 15 and about 25 modifications, between about 20 and 30 modifications, between about 20 and about 40 modifications, between about 25 and about 45 modifications, between about 25 and about 100 modifications, between about 25 and about 45 modifications, between about 35 and about 100 modifications, as compared to a CoV S polypeptide having the amino acid sequence of SEQ ID NO: 2.

In embodiments, the heterogeneous SARS-CoV-2 strain is a SARS-CoV-2 virus comprising a CoV S polypeptide with at least about 70%, at least about 75%, at least about 80%, at least about 85%, at least about 90%, at least about 95%, at least about 96%, at least about 97%, at least about 98%, or at least about 99% identity to a CoV S polypeptide having the amino acid sequence of SEQ ID NO: 2. In embodiments, the heterogeneous SARS-CoV-2 strain is a SARS-CoV-2 virus comprising a CoV S polypeptide with between about 70% and about 99.9% identity to a CoV S polypeptide having the amino acid sequence of SEQ ID NO: 2. In embodiments, the heterogeneous SARS-CoV-2 strain is a SARS-CoV-2 virus comprising a CoV S polypeptide with between about 70% and about 99.5% identity to a CoV S polypeptide having the amino acid sequence of SEQ ID NO: 2. In embodiments, the heterogeneous SARS-CoV-2 strain is a SARS-CoV-2 virus comprising a CoV S polypeptide with between about 90% and about 99.9% identity to a CoV S polypeptide having the amino acid sequence of SEQ ID NO: 1 or SEQ ID NO: 2. In embodiments, the heterogeneous SARS-CoV-2 strain is a SARS-CoV-2 virus comprising a CoV S polypeptide with between about 90% and about 99.8% identity to a CoV S polypeptide having the amino acid sequence of SEQ ID NO: 2. In embodiments, the heterogeneous SARS-CoV-2 strain is a SARS-CoV-2 virus comprising a CoV S polypeptide with between about 95% and about 99.9% identity to a CoV S polypeptide having the amino acid sequence of SEQ ID NO: 2. In embodiments, the heterogeneous SARS-CoV-2 strain is a SARS-CoV-2 virus comprising a CoV S polypeptide with between about 95% and about 99.8% identity to a CoV S polypeptide having the amino acid sequence of SEQ ID NO: 2. In embodiments, the heterogeneous SARS-CoV-2 strain is a SARS-CoV-2 virus comprising a CoV S polypeptide with between about 95% and about 99% identity to a CoV S polypeptide having the amino acid sequence of SEQ ID NO: 2. In embodiments, the heterogeneous SARS-CoV-2 strain has a World Health Organization Label of alpha, beta, gamma, delta, epsilon, eta, iota, kappa, zeta, mu, or omicron. In embodiments, the heterogeneous SARS-CoV-2 strain has a PANGO lineage selected from the group consisting of B.1.1.529; BA.1, BA.1.1, BA.2, BA.3, BA.4, BA.5, B.1.1.7, B.1.351, P.1, B.1.617.2, AY, B.1.427, B.1.429, B.1.525, B.1.526, B.1.617.1, B.1.617.3, P.2, B.1.621, or B.1.621.1. The following document describes the Pango lineage designation and is incorporated by reference herein in its entirety: O'Toole et al. BMC Genomics, 23, 121 (2022).

In embodiments, the heterogeneous SARS-CoV-2 strain has a World Health Organization Label of omicron. In embodiments, the heterogeneous SARS-CoV-2 strain with a World Health Organization Label of omicron has at least 35 modifications compared to the wild-type SARS-CoV-2 S polypeptide of SEQ ID NO: 2. In embodiments, the heterogeneous SARS-CoV-2 strain with a World Health Organization Label of omicron has from 35 to 55, from 35 to 65, from 35 to 75, from 35 to 85, from 35 to 95, or from 35 to 105 modifications compared to the wild-type SARS-CoV-2 S polypeptide of SEQ ID NO: 2. In embodiments, the modifications are selected from the group consisting of T6I, T6R, A14S, A54V, V70A, T82I, G129D, H133Q, K134E, W139R, E143G, F144L, Q170E, I197V, L199I, V200E, V200G, G239V, G244S, G326D, G326H, R333T, L355I, S358F, S358L, S360P, S362F, T363A, D392N, R395S, K404N, N427K, K431T, V432P, G433S, L439R, L439Q, N447K, S464N, T465K, E471A, F473V, F473S, F477S, Q480R, G483S, Q485R, N488Y, Y492H, T534K, T591I, D601G, G626V, H642Y, N645S, N666K, P668H, S691L, N751K, D783Y, N843K, Q941H, N956K, L968F, D1186N, deletion of amino acid 11, deletion of amino acid 12, deletion of amino acid 13, deletion of amino acid 56, deletion of amino acid 57, deletion of amino acid 130, deletion of amino acid 131, deletion of amino acid 132, deletion of amino acid 144, deletion of amino acid 145, deletion of amino acid 198, and insertion of a tripeptide having the amino acid sequence of EPE between amino acids 214 and 215, and combinations thereof.

In embodiments, the CoV S polypeptide of the variant comprises a combination of modifications selected from the group consisting of:
(i) A54V, T82I, G129D, L199I, G326D, S358L, S360P, S362F, K404N, N427K, G433S, S464N, T465K, E471A, Q480R, G483S, Q485R, N488Y, Y492H, T534K, D601G, H642Y, N666K, P668H, N751K, D783Y, N843K, Q941H, N956K, L968F, deletion of amino acid 56, deletion of amino acid 57, deletion of amino acid 130, deletion of amino acid 131, deletion of amino acid 132, deletion of amino acid 198, and insertion of a tripeptide having the amino acid sequence of EPE between amino acids 214 and 215;
(ii) T6I, A14S, G129D, V200G, G326D, S358F, S360P, S362F, T363A, D392N, R395S, K404N, N427K, S464N, T465K, E471A, Q480R, Q485R, N488Y, Y492H, D601G, H642Y, N666K, P668H, N751K, D783Y, Q941H, N956K, deletion of amino acid 11, deletion of amino acid 12, and deletion of amino acid 13;
(iii) T6R, A14S, T82I, G129D, E143G, L199I, G326D, S358L, S360P, K404N, N427K, G433S, S464N, T465K, E471A, Q480R, G483S, Q485R, N488Y, Y492H, T534K, D601G, H642Y, N666K, P668H, N751K, D783Y, N843K, Q941H, N956K, L968F, deletion of amino acid 144, deletion of amino acid 145, deletion of amino acid 198, and insertion of a tripeptide having the amino acid sequence of EPE between amino acids 214 and 215;
(iv) T6I, A14S, G129D, V200G, G326D, S358F, S360P, S362F, T363A, D392N, K404N, N427K, L439Q, S464N, T465K, E471A, Q480R, Q485R, N488Y, Y492H, D601G, H642Y, N666K, P668H, S691L, N751K, D783Y, Q941H, N956K, deletion of amino acid 11, deletion of amino acid 12, and deletion of amino acid 13;
(v) T6I, A14S, G129D, V200G, G326D, S358F, S360P, S362F, T363A, D392N, S464N, T465K, E471A, Q480R, Q485R, N488Y, Y492H, D601G, H642Y, N666K, P668H, D783Y, Q941H, N956K, deletion of amino acid 11, deletion of amino acid 12, and deletion of amino acid 13;
(vi) T6I, A14S, G129D, V200G, G326D, S358F, S360P, S362F, T363A, D392N, R395S, K404N, D601G, H642Y, N645S, N666K, N751K, D783Y, Q941H, N956K, deletion of amino acid 11, deletion of amino acid 12, deletion of amino acid 13, deletion of amino acid 56, and deletion of amino acid 57;
(vii) V3G, T6I, A14S, G129D, V200G, G326D, S358F, S360P, S362F, T363A, D392N, R395S, K404N, L439R, S464N, T465K, E471A, F473V, Q485R, N488Y, Y492H, D601G, G626V, H642Y, N666K, P668H, N751K, D783Y, Q941H, N956K, deletion of amino acid 11, deletion of amino acid 12, deletion of amino acid 13, deletion of amino acid 56, and deletion of amino acid 57;
(viii) V3G, T6I, A14S, G129D, V200G, G326D, S358F, S360P, S362F, T363A, D392N, R395S, K404N, N427K, L439R, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, and deletion of amino acid 57;
(ix) T6I, A14S, G129D, V200G, G326D, S358F, S360P, S362F, T363A, D392N, R395S, K404N, N427K, L439R, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, and deletion of amino acid 57;
(x) T6I, A14S, G129D, K134E, W139R, F144L, I197V, V200G, G244S, G326H, S358F, S360P, S362F, T363A, D392N, R395S, K404N, N427K, G433S, N447K, S464N, T465K, E471A, Q485R, N488Y, Y492H, D601G, H642Y, N666K, P668H, N751K, D783Y, Q941H, N956K, deletion of amino acid 11, deletion of amino acid 12, and deletion of amino acid 13; (xi) T6I, A14S, G129D, K134E, W139R, F144L, I197V, V200G, G244S, G326H, R333T, S358F, S360P, S362F, T363A, D392N, R395S, K404N, N427K, G433S, L439R, N447K, S464N, T465K, E471A, F473S, Q485R, N488Y, Y492H, T591I, D601G, H642Y, N666K, P668H, N751K, D783Y, Q941H, N956K, D1186N, deletion of amino acid 11, deletion of amino acid 12, and deletion of amino acid 13;
(xii) T6I, A14S, G129D, V200G, G326D, R333T, S358F, S360P, S362F, T363A, D392N, R395S, K404N, N427K, L439R, S464N, T465K, E471A, F473V, Q485R, N488Y, Y492H, D601G, H642Y, N645S, N666K, P668H, N751K, D783Y, Q941H, N956K, deletion of amino acid 11, deletion of amino acid 12, deletion of amino acid 13, deletion of amino acid 56, and deletion of amino acid 57;
(xiii) T6I, A14S, G129D, V200G, G326D, R333T, S358F, S360P, S362F, T363A, D392N, R395S, K404N, N427K, L439R, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, and deletion of amino acid 57;
(xiv) T6I, A14S, V70A, G129D, H133Q, Q170E, V200E, G239V, G326H, R333T, L355I, S358F, S360P, S362F, T363A, D392N, R395S, K404N, N427K, V432P, G433S, N447K, S464N, T465K, E471A, F473S, F477S, Q485R, N488Y, Y492H, D601G, H642Y, N666K, P668H, N751K, D783Y, Q941H, N956K, deletion of amino acid 11, deletion of amino acid 12, deletion of amino acid 13, and deletion of amino acid 131;
(xv) T6I, A14S, G129D, H133Q, Q170E, V200E, G326H, R333T, L355I, S358F, S360P, S362F, T363A, D392N, R395S, K404N, N427K, V432P, G433S, N447K, S464N, T465K, E471A, F473S, F477S, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, deletion of amino acid 57, and deletion of amino acid 131;
(xvi) T6I, A14S, G129D, V200G, G326D, R333T, S358F, S360P, S362F, T363A, D392N, R395S, K404N, N427K, K431T, L439R, N447K, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, and deletion of amino acid 57;
(xvii) T6I, A14S, G129D, V200G, G326D, S358F, S360P, S362F, T363A, D392N, R395S, K404N, N427K, K431T, L439R, N447K, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, and deletion of amino acid 57; and
(xviii) T6I, A14S, G129D, V200G, G326D, R333T, S358F, S360P, S362F, T363A, D392N, R395S, K404N, N427K, L439R, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, deletion of amino acid 57, and deletion of amino acid 131;
(xix) deletion of amino acid 56, deletion of amino acid 57, and deletion of amino acid 131, N488Y, A557D, D601G, P668H or P668R, T7031, S969A, and D1105H;
(xx) D67A, K404N, E471K, N488Y, D601G, and A688V;
(xxi) D67A, D202G, L229H, K404N, E471K, N488Y, D601G, and A688V;
(xxii) D67A, D202G, deletion of 1, 2, or 3 amino acids of amino acids 228-230, K404N, E471K, N488Y, D601G, and A688V;
(xxiii) D67A, L229H, R233I, N488Y, K404N, E471K, D601G, and A688V;
(xxiv) L5F, T7N, P13S, D125Y, R177S, K404T, E471K, N488Y, D601G, H642Y, T1014I, and V1163F;
(xxv) W139C and L439;
(xxvi) deletion of amino acid 144, deletion of amino acid 145, T6R, E143G, L439R, T465K, D601G, P668R, and D937N;
(xxvii) deletion of amino acid 144, deletion of amino acid 145, T6R, G129D, E143G, L439R, T465K, D601G, P668R, and D937N;
(xxviii) deletion of amino acid 144, deletion of amino acid 145, T6R, T82I, G129D, Y132H, E143G, A209V, K404N L439R, T465K, D601G, P668R, and D937N;
(xxix) deletion of amino acid 144, deletion of amino acid 145, T6R, G129D, E143G, W245I, K404N, N426K, L439R, T465K, E471K, N488Y, D601G, P668R, and D937N;
(xxx) deletion of amino acid 144, deletion of amino acid 145, T6R, W51H, H53W, G129D, E143G, D200V, L201R, W245I, K404N, N426K, L439R, T465K, E471K, N488Y, D601G, P668R, and D937N;
(xxxi) deletion of amino acid 144, deletion of amino acid 145, T6R, G129D, E143G, K404N, L439R, T465K, E471Q, D601G, P668R, and D937N;
(xxxii) Q39R, A54V, E471K; D601G, Q664H, F875L, and deletion of 1, 2, 3, or 4 of amino acids 56, 57, 131, 132;
(xxxiii) T82I, D240G, E471K, D601G, and A688V;
(xxxiv) L439R, E471Q, D601G, P668R, and Q1058H;
(xxxv) G62V, T63I, R233N, L439Q, F477S, D601G, T846N, and deletion of 1, 2, 3, 4, 5, or 6 of amino acids 234-240;
(xxxvi) T82I, Y131S, Y132N, R333K, E471K, N488Y, D601G, P668H, and D937N; and
(xxxvii) G129D, G326D, S360P, S362F, K404N, N427K, T465K, E471A or E471K, Q480K or Q480R, Q485R, N488Y, Y492H, D601G, H642Y, N666K, P668H, N751K, D783Y, Q941H, and N953K, wherein the amino acids of the CoV S glycoprotein are numbered with respect to a polypeptide having the sequence of SEQ ID NO: 2.

As used herein, the term “NVX-CoV2373” refers to a vaccine composition comprising the BV2373 Spike glycoprotein (SEQ ID NO: 3) and Fraction A and Fraction C iscom matrix (e.g., MATRIX-M™).

### Methods for Evaluating the Immunogenicity of Vaccine Compositions Against SARS-CoV-2

Provided herein is a method for determining if a biological sample contains antibodies that inhibit binding of a SARS-CoV-2 S glycoprotein to human angiotensin-converting enzyme 2 (hACE2), comprising: (a) measuring binding of a SARS-CoV-2 S glycoprotein to hACE2 after exposure to a biological sample by: (i) providing a surface coated with the SARS-CoV-2 S glycoprotein; (ii) exposing the surface to the biological sample; (iii) exposing the surface to hACE2; and (iv) detecting hACE2 that is bound to the surface; (b) measuring binding of a SARS-CoV-2 S glycoprotein to hACE2 in the absence of the biological sample of (a) by: (i) providing a surface coated with the SARS-CoV-2 S glycoprotein; (ii) exposing the surface to hACE2; and (iii) detecting hACE2 that is bound to the surface; and (c) comparing binding of the SARS-CoV-2 S glycoprotein to hACE2 in (a) and (b); wherein a biological sample contains antibodies that inhibit binding of the SARS-CoV-2 S glycoprotein to hACE2 if the amount of bound hACE2 in (a) is less than the amount of bound hACE2 in (b). In embodiments, step a(ii) is performed before step a(iii). In embodiments, step a(iii) is performed before step a(ii).

In embodiments, the methods further comprise washing the solid surface. In embodiments, the methods comprise contacting the plate with a blocking buffer. A blocking buffer is a solution that removes the possibility of non-specific binding to the plate.

In embodiments, the methods may be utilized to detect antibody isotypes selected from the group consisting of IgA, IgG, IgM, IgD, and IgE.

### SARS-CoV-2 S Glycoproteins for Use in the Methods

In embodiments, suitable SARS-CoV-2 S glycoproteins for use in the methods described herein include the SARS-CoV-2 S glycoproteins associated with Protein Data Bank (PDB) IDs of any one of: 6LVN,6LZG,6M0J,6M17,6M1V,6VSB,6VW1,6VXX,6VYB,6W41,6WPS,6WPA, 6X2B,6X2C,6X45,6X6P,6X79,6XC2,6XC3,6XC4,6XC7,6XCM,6XCN,6XDG,6XE1,6XEY, 6XF5,6XF6,6XKL,6XKP,6XKQ,6XLU,6XM0,6XM3,6XM4,6XM5,6XR8,6XRA,6XS6,6YBB, 6YLA,6YM0,6YOR,6YZ5,6YZ7,6Z2M,6Z43,6Z97,6ZB4,6ZB5,6ZBP,6ZCZ,6ZDG,6ZDH, 6ZER,6ZFO,6ZGE,6ZGG,6ZGH,6ZGI,6ZH9,6ZHD,6ZLR,6ZOW,6ZOX,6ZOY,6ZOZ, 6ZP0,6ZP1,6ZP2,6ZP5,6ZP7,6ZWV,6ZXN,7A25,7A29,7A4N,7A5R,7A5S,7A91,7A92,7A93, 7A94,7A95,7A96,7A97,7A98,7AD1,7AKD,7B0B,7B14,7B17,7B18,7B27,7B30,7B62,7BEH, 7BEI,7BEJ,7BEK,7BEL,7BEM,7BEN,7BEO,7BEP,7BH9,7BNM,7BNN,7BNO,7BNV, 7BWJ,7BYR,7BZ5,7C01,7C2L,7C53,7C8D,7C8J,7C8V,7C8W,7CAB,7CAC,7CAH,7CAI,7CAK, 7CAN,7CDI,7CDJ,7CH4,7CH5,7CHB,7CHC,7CHE,7CHF,7CHH,7CHO,7CHP,7CHS, 7CJF,7CM4,7CN9,7COT,7CT5,7CWL,7CWM,7CWN,7CWO,7CWS,7CWT,7CWU,7CYH, 7CYP,7CYV,7CZP,7CZQ,7CZR,7CZS,7CZT,7CZU,7CZV,7CZW,7CZX,7CZY,7CZZ,7D00, 7D03,7D0B,7D0C,7D0D,7D2Z,7D30,7D4G,7D6I,7DCC,7DCX,7DD2,7DD8,7DDD,7DDN, 7DEO,7DET,7DEU,7DF3,7DF3,7DF4,7DHX,7DJZ,7DK0,7DK2,7DK3,7DK4,7DK,7DK6,7DK7, 7DMU,7DPM,7DQA,7DTE,7DWX,7DWY,7DWZ,7DX0,7DX1,7DX2,7DX3,7DX4,7DX5, 7DX6,7DX7,7DX8,7DX9,7DZW,7DZX,7DZY,7E23,7E39,7E3B,7E3C,7E3J,7E3K,7E3 L,7E30,7E50,7E5R,7E5S,7E5Y,7E7B,7E7D,7E7X,7E7Y,7E86,7E88,7E8C,7E8F,7E8M,7E 9N,7E90,7E9P,7E9Q,7E9H, 7EDI,7EDJ,7EFP,7EFR,7EH5,7EJ4,7EJ5,7EJL,7EJY,7EJZ,7EK0,7EK6,7EKC,7EKE,7EKF, 7EKG,7EKH,7ENF,7ENG,7EPX,7EY0,7EY4,7EY5,7EYA,7EZV,7F0X,7F12,7F15,7F3Q, 7F46,7F5H,7F5R,7F62,7F63,7F6Y,7F6Z,7F7E,7F7H,7FAE,7FAF,7FA1, 7FB3,7FB4,7FBJ,7FBK,7FC5,7FCD,7FCE,7FCP,7FCQ,7FDG,7FDH,7FDI,7FDK,7FEM, 7FET,7FG2,7FG3,7FG7,7FH0,7FJC,7FJN,7FJO,7FJS,7JJC,7JJI,7JJJ,7JMO,7JMP,7JMW,7JV2, 7JV4,7JV6,7JVA,7JVB,7JVC,7JW0,7JWB,7JWY,7JX3,7JZL,7JZM,7JZN,7JZU,7K43,7K45, 7K4N,7K8M,7K8S,7K8U,7K8V,7K8W,7K8Y,7K8,7K90,7K9H,7K9I,7K9J, K9J,7K9K,7K9Z,7KDG,7KDH,7KDI,7KDJ,7KDK,7KDL,7KE4,7KE6,7KE7,7KE8,7KE9,7KEA, 7KEB,7KEC,7KFV,7KFW,7KFX,7KFY,7KGJ,7KGK,7KJ2,7KJ3,7KJ4,7KJ5,7KKK,7KKL, 7KL9,7KLG,7KLH,7KLW,7KM5,7KMB,7KMG,7KMH,7KMI,7KMK,7KML,7KMS, 7KMZ,7KN3,7KN4,7KN5,7KN6,7KN7,7KNB,7KNE,7KNH,7KNI,7KQE,7KRQ,7KRR,7KRS, 7KS9,7KSG,7KXJ,7KXK,7KZB,7L02,7L06,7L09,7LON,7L2C,7L2D,7L2E,7L2F,7L3N, 7L4Z,7L56,7L57,7L58,7L5B,7L7D,7L7E,7L7F,7L7K,7LAA,7LAB,7LC8,7LCN,7LD1,7LDJ, 7LJR,7LM8,7LO4,7LOP,7LQ7,7LQV,7LQW,7LRS,7LRK, 7LWL,7LWM,7LWN,7LWO,7LWP,7LWQ,7LWS,7LWT,7LWU,7LWV,7LWW,7LX5,7LXW, 7LXX,7LXY,7LXZ,7LYO,7LY2,7LY3,7LYK,7LYL,7LYM,7LYN,7LYO,7LYP,7LYQ, 7M0J,7M3I,7M42,7M53,7M6D,7M6E,7M6F,7M6G,7M6H,7M6I,7M71,7M7B,7M7W, 7M8J,7M8K,7M8S,7M8T,7M8U,7MDW,7ME7,7MEJ,7MF1,7MFU,7MJG,7MJH,7MJI,7MJJ, 7MJK,7MJL,7MJM,7MJN,7MKL,7MKM,7MLZ,7MMO,7MMO,7MSQ,7MTC,7MTD,7MTE, 7MW2,7MW3,7MW4,7MW5,7MW6,7MY2,7MY3,7MY8,7MZF,7MZG,7MZH,7MZI,7MZJ, 7MZK,7MZL,7MZM,7MZN,7N0G,7N0H,7N1A,7N1B,7N1E,7N1F,7N1Q,7N1T,7N1U,7N1V, 7N1W,7N1X,7N1Y,7N3I,7N4I,7N4J,7N4L,7N4M,7N5H,7N62,7N64,7N6D,7N6E,7N8H, 7N8I,7N9A,7N9B,7N9C,7N9E,7N9T,7NAB,7ND3,7ND4,7ND5,7ND6,7ND7,7ND8,7ND9, 7NDA,7NDB,7NDC,7NDD,7NEG,7NEH,7NKT,7NLL,7NP1,7NS6,7NT9,7NTA,7NTC,7NX6, 7NX7,7NX8,7NX9,7NXA,7NXB,7NXC,7OAN,7OAO,7OAP,7OAQ,7OAU,7OAY,7OD3, 7ODL,7OLZ,7OR9,7ORA,7ORB,7OWX,7P19,7P3D,7P40,7P5G,7P5Q,7P5S,7P77,7P78, 7P79,7P7A,7P7B,7PBE,7PHG,7PQY,7PQZ,7PR0,7PRY,7PRZ,7PS0,7PS1,7PS2,7PS4,7PS5, 7PS6,7PS7,7Q0A,7Q0G,7Q0H,7Q0I,7Q1Z,7Q3Q,7Q3R,7Q6E,7Q9F,7Q9G,7Q9I,7Q9J,7Q9K,7Q9M,7Q9P,7QEZ,7QF0,7QF1,7QNW,7QNX,7QNY,7QO7,7QO9,7QTI,7QTJ,7QTK,7QUR, 7QUS,7R0Z,7R10,7R11,7R12,7R13,7R14,7R15,7R16,7R17,7R18,7R19,7R1A,7R1B,7R40, 7R4I,7R4Q,7R4R,7R6W,7R6X,7R7N,7R8L,7R8M,7R8N,7R80,7R95,7RA8,7RAL,7RAQ, 7RBU,7RBV,7RBY,7RKU,7RKV,7RNJ,7RPV,7RQ6,7RR0,7RTD,7RTR,7RU1,7RU2,7RU3, 7RU4,7RU5,7RU8,7RW2,7RXD,7RZQ,7RZR,7RZ S,7RZ0D, 7S0E,7S3N,7S4S,7S5P,7S5Q,7S5R,7S6I,7S6J,7S6K,7S6L,7S83,7SA2,7SBK,7SBL,7SBO, 7SBP,7SBQ,7SBR,7SBS,7SBT,7SBU,7SC1,7SD5,7SI2,7SIS,7SIZ,7SJ0,7SJS,7SKZ,7SL5, 7SN0,7SN2,7SN3,7SO9,7SOA,7SOB,7SOC,7SOD,7SOE,7SOF,7SPO,7SPP,7SWN,7SWO, 7SWP,7SWW,7SWX,7SXR,7SXS,7SXT,7SXU,7SXV,7SXW,7SXX,7SXY,7SXZ,7SY0,7SY1, 7SY2,7SY3,7SY4,7SY5,7SY6,7SY7,7SY8,7T01,7T3M,7T67,7T72,7T7B,7T9J,7T9K,7T9L, 7TAS,7TAT,7TB4,7TB8,7TBF,7TCA,7TCC,7TCQ,7TEI,7TEW,7TEX,7TEY,7TEZ,7TF0, 7TF1,7TF2,7TF3,7TF4,7TF5,7TF8,7TGE,7TGW,7TGX,7TGY,7THE,7THK,7THT,7TIK, 7TL1,7TL9,7TLA,7TLB,7TLC,7TLD,7TLT,7TLY,7TM0,7TN0,7TNW,7TO4,7TOU,7TOV, 7TOW,7TOX,7TOY,7TOZ,7TP0,7TP1,7TP2,7TP3,7TP4,7TP7,7TP8,7TP9,7TPA,7TPC,7TPE, 7TPF,7TPH,7TPK,7TPL,7TPR,7TYZ,7TZ0,7U09,7UA,7U0D,7U0E,7U0N,7U0P,7U0Q, 7U0X,7U1R,7U2D,7U2E,7U8E,7U90,7U9P,7UAP,7UAQ,7UAR,7UB0,7UB5,7UB6,7UFK, 7UFL,7UHC,7UL0,7UM2,7UPL,7UR1,7URQ,7URS,7UZ4,7UZ5,7UZ6,7UZ7,7UZ8,7UZ9, 7UZA,7UZB,7UZC,7UZD,7V20,7V22,7V23,7V24,7V26,7V27,7V2A,7V76,7V77,7V78,7V79, 7V7A,7V7D,7V7E,7V7F,7V7G,7V7H,7V7I,7V7J,7V7N,7V7O,7V7P,7V7Q,7V7R,7V7S, 7V7T,7V7U,7V7V,7V7Z,7V80,7V81,7V84,7V85,7V86,7V87,7V88,7V89,7V8A, 7V8B,7V8C,7VHH,7VHJ,7VHK,7VHL,7VHM,7VHN,7VMU,7VNB,7VNC,7VND,7VNE, 7VOA,7VQ0,7VRV,7VRW,7VX1,7VX4,7VX5,7VX9,7VXA,7VXB,7VXC,7VXD,7VXE, 7VXF,7VXI,7VXK,7VXM,7VYR,7VZT,7W1S,7W6U,7W8S,7W92,7W94,7W99,7W9B,7W9C, 7W9E,7W9F,7WA1,7WB5,7WBL,7WBP,7WBQ,7WBZ,7WCD,7WCH,7WCK,7WCP, 7WCR,7WCU,7WCZ,7WD0,7WD1,7WD2,7WD7,7WD8,7WD9,7WDF,7WE7,7WE8,7WE9, 7WEA,7WEB,7WEC,7WED,7WEE,7WEF,7WEV,7WG6,7WG7,7WG8,7WG9,7WGB,7WGC, 7WGV,7WGX,7WGY,7WGZ,7WH8,7WHB,7WHD,7WHH,7WHI,7WHJ,7WHK,7WHZ, 7WJY,7WJZ,7WK0,7WK2,7WK3,7WK4,7WK5,7WK6,7WK8,7WK9,7WKA,7WLC,7WM0, 7WN2,7WNB,7WNM,7WO4,7WO5,7WO7,7WOA,7WOB,7WOC,7WOG,7WON,7WOP, 7WOQ,7WOR,7WOS,7WOU,7WOV,7WOW,7WPO,7WP1,7WP2,7WP5,7WP6,7WP8, 7WP9,7WPA,7WPB,7WPC,7WPD,7WPE,7WPF,7 WPH,7WQV,7WR8,7WRH,7WRI,7WRJ, 7WRV,7WS0,7WS1,7WS2,7WS3,7WS4,7WS5,7WS6,7WS7,7WS8,7WS9,7WSA,7WSE,7WSH, 7WSK,7WT7,7WT8,7WT9,7WTF,7WTG,7WTH,7WTI,7WTJ,7WTK,7WUE,7WUH, 7WVL,7WVP,7WVQ,7WWI,7WWJ,7WWK,7WWL,7WWM,7WXZ,7WZ1,7WZ2,7X1M,7X25, 7X2H,7X2K,7X2L,7X2M,7X63,7×66,7×6A,7X7D,7X7E,7X7N,7X7X8Y, 7X8Z,7X90,7X91,7X92,7X93,7X94,7X95,7X96,7X9E,7XA7,7XAZ,7XB0,7XB1,7XBY, 7XCH,7XCI,7XCK,7XCO,7XCP,7XCZ,7XD2,7XDA,7XDB,7XDK,7XDL,7XEG,7XEI,7XH8, 7XIC,7XID,7XIK,7XIL,7XIW,7XIX,7XIY,7XIZ,7XJ6,7XJ8,7XJ9,7XMX,7XMZ,7XNQ, 7XNR,7XNS,7XO4,7XO5,7XO6,7XO7,7XO8,7XO9,7XOA,7XOB,7XOC,7XOD,7XRP,7XS8, 7XSA,7XSB,7XSC,7XST,7XTZ,7XU0,7XU1,7XU2,7XU3,7XU4,7XU5,7XU6,7XUA, 7XXL,7Y0C,7Y0V,7Y1Y,7Y1Z,7Y42,7Y6D,7Y6K,7Y6L,7Y6N,7Y75,7Y76,7Y7J,7Y7K,7Y8J, 7Y9N,7Y9S,7Y9Z,7YA0,7YA1,7YAD,7YBI,7YBJ,7YC5,7YCK,7YCL,7YCN,7YCO,7YD1, 7YDI,7YDY,7YE5,7YE9,7YEG,7YH6,7YH7,7YHW,7YJ3,7YKJ,7YOW,7YQU, 7YQV,7YQW,7YQX,7YQY,7YQZ,7YR0,7YR1,7YR2,7YR3,7YTN,7YUE,7YV8,7YVE, 7YVF,7YVG,7YVH,7YVI,7YVJ,7YVK,7YVL,7YVM,7YVN,7YVO,7YVP,7YVU,7Z0X,7Z0Y, 7Z1A,7Z1B,7Z1C,7Z1D,7Z1E,7Z3Z,7Z6V,7Z7X,7Z85,7Z86,7Z80,7Z9Q,7Z9R,7ZBU, 7ZCE,7ZCF,7ZDQ,7ZF3,7ZF4,7ZF5,7ZF7,7ZF8,7ZF9,7ZFA,7ZFB,7ZFC,7ZFD,7ZFE,7ZJL, 7ZR2,7ZR7,7ZR8,7ZR9,7ZRC,7ZRV,7ZSD,7ZSS,7ZXU,8A99,8AAA,8AQS,8AQU, 8AQV,8AQW,8BBN,8BBO,8BCZ,8BE1,8BEV,8BGG,8BH5,8BON,8BSE,8BSF,8C1V,8C3V, 8C8P,8CIM,8CSA,8CSJ,8CWI,8CWK,8CWU,8CWV,8CXN,8CXQ,8CY6,8CY7,8CY9, 8CYA,8CYB,8CYC,8CYD,8CYJ,8CZI,8D0Z,8D36,8D47,8D48,8D55,8D56,8D5A,8D6Z, 8D8Q,8D8R,8DAD,8DAO,8DCC,8DCE,8DF5,8DGU,8DI5,8DLI,8DLJ,8DLK,8DLL,8DLM, 8DLN,8DLO,8DLP,8DLQ,8DLR,8DLS,8DLM0, 8DM1, 8DM2,8DM3, 8DM4,8DM5,8DM6,8DM7,8DM8,8DM9,8DMA,8DNN,8DT3,8DT8, 8DTR,8DTA,8DZH,8DZI,8E1G,8EL2,8ELH,8ELJ,8ELO,8ELP,8ELQ,8EOO,8EPN,8EPP,8EPQ,8ERQ, 8ERR,8FOG,8FOH,8FA1,8FA2,8FEZ,8FU7,8FU8,8FU9,8GB0,8GB5,8GB6,8GB7,8GB8,8GJM, 8GJN,8GOM,8GON,8GOU,8GPY,8GRY,8GS6,8GS9,8GTO,8GTP,8GTQ,8GX9,8GZ5, 8GZZ,8H00,8H01,8H06,8H07,8H08,8H3D,8H3E,8H3M,8H3N,8H5C,8HC2,8HC3, 8HC4,8HC5, 8HC6,8HC7,8HC8,8HC9,8HCA,8HCB,8HEB,8HEC,8HED,8HHX,8HHY,88HHZ,8HN6, 8HN7,8I5H,8I5I,8IDN,8IF2,8IOS,8IOT,8IOV,8ITU,8J1Q,8J26, or 8SMT. Each of these PDB IDs is incorporated by reference herein in its entirety. The amino acid sequence of the SARS-CoV-2 S glycoprotein associated with each entry may be accessed by downloading the FASTA file associated with the PDB ID at www.rcsb.org.

In embodiments, the SARS-CoV-2 S glycoprotein is a wild-type SARS-CoV-2 S glycoprotein, or a SARS-CoV-2 S glycoprotein from a SARS-CoV-2 variant thereof. In embodiments, the variant of SARS-CoV-2 is a B.1.1.7 SARS-CoV-2 strain; a B.1.351 SARS-CoV-2 strain; a P.1 SARS-CoV-2 strain; a Cal.20C SARS-CoV-2 strain; a B.1.617.2 SARS-CoV-2 strain; a B.1.525 SARS-CoV-2 strain; a B.1.526 SARS-CoV-2 strain; a B.1.617.1 SARS-CoV-2 strain; a C.37 SARS-CoV-2 strain; a B.1.621 SARS-CoV-2 strain; or a B.1.1.529 SARS-CoV-2 strain. In embodiments, the SARS-CoV-2 S glycoprotein contains a transmembrane domain.

The wild-type SARS-CoV-2 S glycoprotein contains a furin cleavage site, RRAR (SEQ ID NO: 67) at positions 669-672 of the SARS-CoV-2 S glycoprotein of SEQ ID NO: 2. In embodiments, the SARS-CoV-2 S glycoprotein has an inactive furin cleavage site. In embodiments, the inactive furin cleavage site has the amino acid sequence of any one of SEQ ID NOS: 68-97. In embodiments, the amino acid sequence of the inactive furin cleavage site is GG. In embodiments, non-limiting examples of SARS-CoV-2 S glycoproteins with an inactive furin cleavage site of GG include glycoproteins of SEQ ID NOS: 26-28 and 30.

In embodiments, the amino acid sequence of the inactive furin cleavage site is QQAQ (SEQ ID NO: 68). In embodiments, non-limiting examples of SARS-CoV-2 S glycoproteins with an inactive furin cleavage site of QQAQ (SEQ ID NO: 68) include glycoproteins of SEQ ID NOS: 3, 5-13, 15, 17-19, 21, 23-25, 29, and 31-66.

In embodiments, one or more of the amino acids comprising the native furin cleavage site is mutated to any natural amino acid. In embodiments, one or more of the amino acids comprising the native furin cleavage site is deleted.

In embodiments, one or more of the amino acids comprising the native furin cleavage site is mutated to glutamine. In embodiments, 1, 2, 3, or 4 amino acids may be mutated to glutamine. In embodiments, one of the arginines comprising the native furin cleavage site is mutated to glutamine. In embodiments, two of the arginines comprising the native furin cleavage site are mutated to glutamine. In embodiments, three of the arginines comprising the native furin cleavage site are mutated to glutamine.

In embodiments, one or more of the amino acids comprising the native furin cleavage site, is mutated to alanine. In embodiments, 1, 2, 3, or 4 amino acids may be mutated to alanine. embodiments, one of the arginines comprising the native furin cleavage site is mutated to alanine. In embodiments, two of the arginines comprising the native furin cleavage site are mutated to alanine. In embodiments, three of the arginines comprising the native furin cleavage site are mutated to alanine.

In embodiments, one or more of the amino acids comprising the native furin cleavage site is mutated to glycine. In embodiments, 1, 2, 3, or 4 amino acids may be mutated to glycine. In embodiments, one of the arginines of the native furin cleavage site is mutated to glycine. In embodiments, two of the arginines comprising the native furin cleavage site are mutated to glycine. In embodiments, three of the arginines comprising the native furin cleavage site are mutated to glycine.

In embodiments, one or more of the amino acids comprising the native furin cleavage site, is mutated to asparagine. For example 1, 2, 3, or 4 amino acids may be mutated to asparagine. In embodiments, one of the arginines comprising the native furin cleavage site is mutated to asparagine. In embodiments, two of the arginines comprising the native furin cleavage site are mutated to asparagine. In embodiments, three of the arginines comprising the native furin cleavage site are mutated to asparagine.

In embodiments, the active furin cleavage site (SEQ ID NO: 67) of the SARS-CoV-2 S glycoproteins described herein is replaced with an inactivated furin cleavage site of the table below.

### Inactivated Furin Cleavage Sites

| Amino Acid Sequence of | Active or Inactive |
|---|---|
| Furin Cleavage Site | Furin Cleavage Site |
| RRAR (SEQ ID NO: 67) | Active |
| QQAQ (SEQ ID NO: 68) | Inactive |
| QRAR (SEQ ID NO: 69) | Inactive |
| RQAR (SEQ ID NO: 70) | Inactive |
| RRAQ (SEQ ID NO: 71) | Inactive |
| QQAR (SEQ ID NO: 72) | Inactive |
| RQAQ (SEQ ID NO: 73) | Inactive |
| QRAQ (SEQ ID NO: 74) | Inactive |
| NNAN (SEQ ID NO: 75) | Inactive |
| NRAR (SEQ ID NO: 76) | Inactive |
| RNAR (SEQ ID NO: 77) | Inactive |
| RRAN (SEQ ID NO: 78) | Inactive |
| NNAR (SEQ ID NO: 79) | Inactive |
| RNAN (SEQ ID NO: 80) | Inactive |
| NRAN (SEQ ID NO: 81) | Inactive |
| AAAA (SEQ ID NO: 82) | Inactive |
| ARAR (SEQ ID NO: 83) | Inactive |
| RAAR (SEQ ID NO: 84) | Inactive |
| RRAA (SEQ ID NO: 85) | Inactive |
| AAAR (SEQ ID NO: 86) | Inactive |
| RAAA (SEQ ID NO: 87) | Inactive |
| ARAA (SEQ ID NO: 88) | Inactive |
| GGAG (SEQ ID NO: 89) | Inactive |
| GRAR (SEQ ID NO: 90) | Inactive |
| RGAR (SEQ ID NO: 91) | Inactive |
| RRAG (SEQ ID NO: 92) | Inactive |
| GGAR (SEQ ID NO: 93) | Inactive |
| RGAG (SEQ ID NO: 94) | Inactive |
| GRAG (SEQ ID NO: 95) | Inactive |
| GSAS (SEQ ID NO: 96) | Inactive |
| GSGA (SEQ ID NO: 97) | Inactive |

In embodiments, the SARS-CoV-2 S glycoproteins contain a mutation at Lys-973 of the native SARS-CoV-2 S glycoprotein (SEQ ID NO: 2). In embodiments, Lys-973 is mutated to any natural amino acid. In embodiments, Lys-973 is mutated to proline. In embodiments, Lys-973 is mutated to glycine.

In embodiments, the SARS-CoV-2 S glycoproteins contain a mutation at Val-974 of the native SARS-CoV-2 S glycoprotein (SEQ ID NO: 2). In embodiments, Val-974 is mutated to any natural amino acid, as compared to the SARS-CoV-2 S glycoprotein of SEQ ID NO: 2. In embodiments, Val-974 is mutated to proline. In embodiments, Val-974 is mutated to glycine, as compared to the SARS-CoV-2 S glycoprotein of SEQ ID NO: 2.

In embodiments, the SARS-CoV-2 S glycoproteins contain a mutation at Lys-973 and Val-974 of the native CoV Spike (S) polypeptide (SEQ ID NO: 2). In embodiments, Lys-973 and Val-974 are mutated to any natural amino acid, as compared to the SARS-CoV-2 S glycoprotein of SEQ ID NO: 2. In embodiments, Lys-973 and Val-974 are mutated to proline, as compared to the SARS-CoV-2 S glycoprotein of SEQ ID NO: 2. Non-limiting examples of SARS-CoV-2 S glycoproteins where amino acids Lys-973 and Val-974 are mutated to proline include glycoproteins of SEQ ID NOS: 3-13, 20, and 22-60.

In embodiments, the SARS-CoV-2 S glycoprotein has at least 80%, at least 81% at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of SEQ ID NO: 1, SEQ ID NO: 2, or SEQ ID NO: 3.

In embodiments, the SARS-CoV-2 S glycoprotein has at least 80%, at least 81% at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 3-66.

Providing a Surface Coated with a SARS-CoV-2 S Glycoprotein

In embodiments, the methods described herein require providing a surface coated with a SARS-CoV-2 S glycoprotein. In embodiments, the surface is a chip or a microplate. In embodiments, the microplate comprises polystyrene. In embodiments, the microplate is 96-well or 384-well polystyrene plate. In embodiments, the surface is a microplate that is filled with a solution.

### Exposing the Surface to a Biological Sample

In embodiments, the surface is exposed to a biological sample. In embodiments, the surface is exposed to the biological sample for from 10 minutes to about 72 hours. In embodiments, the surface is exposed to the biological sample for about 10 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 51 hours, about 52 hours, about 53 hours, about 54 hours, about 55 hours, about 56 hours, about 57 hours, about 58 hours, about 59 hours, about 60 hours, about 61 hours, about 62 hours, about 63 hours, about 64 hours, about 65 hours, about 66 hours, about 67 hours, about 68 hours, about 69 hours, about 70 hours, about 71 hours, or about 72 hours, including any range or any value therebetween. In embodiments, the surface is exposed to the biological sample for about 1 hour or 2 hours. In embodiments, the surface is exposed to the biological sample at a temperature from 2-8° C. or from 8-37° C. In embodiments, the surface is exposed to the biological sample at a temperature of 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., or about 37° C.

In embodiments, the biological sample is saliva, a nasopharyngeal swab, sputum, saliva, urine, a fecal sample, cerebrospinal fluid, synovial fluid, serum, blood, or plasma. In embodiments, the biological sample is from a patient that has previously had COVID-19. In embodiments, the biological sample is from a patient that has been administered an immunogenic composition against a SARS-CoV-2 virus or a variant thereof.

Exposing the Surface to hACE2

In embodiments, hACE2 comprises a polypeptide with at least 80%, at least 81% at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of SEQ ID NOS: 98 or 99.

In embodiments, the hACE2 is attached to a tag. In embodiments, the hACE2 is covalently attached to a tag. In embodiments, the hACE2 is non-covalently attached to a tag. In some aspects, the tag is useful for detection of hACE2 binding. In embodiments, the tag is a His tag. In embodiments, the tag contains an epitope. For example, the tag may be a polyglutamate tag, a FLAG-tag, a HA-tag, a polyHis-tag (having about 5-10 histidines) (SEQ ID NO: 100), a hexahistidine tag (SEQ ID NO: 101), a 7X-His-tag (having seven histidines) (SEQ ID NO: 102), an 8X-His-tag (having eight histidines) (SEQ ID NO: 103), a Myc-tag, a Glutathione-S-transferase-tag, a Green fluorescent protein-tag, Maltose binding protein-tag, a Thioredoxin-tag, or an Fc-tag. In embodiments, the tag is a protease cleavage site. Non-limiting examples of protease cleavage sites include the HRV3C protease cleavage site, chymotrypsin, trypsin, elastase, endopeptidase, caspase-1, caspase-2, caspase-3, caspase-4, caspase-5, caspase-6, caspase-7, caspase-8, caspase-9, caspase-10, enterokinase, factor Xa, Granzyme B, TEV protease, and thrombin. In embodiments, the protease cleavage site is an HRV3C protease cleavage site.

In embodiments, the surface is exposed to hACE2 for from 10 minutes to about 72 hours. In embodiments, the surface is exposed to hACE2 for about 10 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 51 hours, about 52 hours, about 53 hours, about 54 hours, about 55 hours, about 56 hours, about 57 hours, about 58 hours, about 59 hours, about 60 hours, about 61 hours, about 62 hours, about 63 hours, about 64 hours, about 65 hours, about 66 hours, about 67 hours, about 68 hours, about 69 hours, about 70 hours, about 71 hours, or about 72 hours, including any range or any value therebetween.

Detecting hACE2 that is Bound to the Surface

In embodiments, the methods comprise detecting hACE2 that is bound to the surface. In embodiments, hACE2 is detected by contacting the surface with an antibody that binds to hACE2. In embodiments, the surface is contacted with an antibody that binds to hACE2 for about 10 minutes to about 72 hours. In embodiments, the surface is contacted with an antibody that binds to hACE2 for about 10 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 51 hours, about 52 hours, about 53 hours, about 54 hours, about 55 hours, about 56 hours, about 57 hours, about 58 hours, about 59 hours, about 60 hours, about 61 hours, about 62 hours, about 63 hours, about 64 hours, about 65 hours, about 66 hours, about 67 hours, about 68 hours, about 69 hours, about 70 hours, about 71 hours, or about 72 hours, including any range or any value therebetween.

In embodiments, the antibody is an anti-polyhistidine tag antibody. In embodiments, the antibody is tagged with horseradish peroxidase. In embodiments, the antibody is tagged with alkaline phosphatase. In embodiments, detecting hACE2 comprises (i) contacting the surface with an antibody that binds to hACE2, wherein the antibody is tagged with horseradish peroxidase; and (ii) contacting the surface with 3,3′,5,5′-tetramethylbenzidine substrate. In embodiments, detecting comprises determining the absorbance of the surface. In embodiments, detecting comprises determining the absorbance of the surface at a wavelength from 400 nm to about 650 nm. In embodiments, detecting comprises determining the absorbance of the surface at a wavelength of about 400 nm, about 410 nm, about 420 nm, about 430 nm, about 440 nm, about 450 nm, about 460 nm, about 470 nm, about 480 nm, about 490 nm, about 500 nm, about 510 nm, about 520 nm, about 530 nm, about 540 nm, about 550 nm, about 560 nm, about 570 nm, about 580 nm, about 590 nm, about 600 nm, about 610 nm, about 620 nm, about 630 nm, about 640 nm, or about 650 nm, including all values and ranges therebetween. In embodiments, detecting comprises determining the absorbance of the surface at a wavelength of about 450 nm.

In embodiments, the methods used herein can be used to evaluate the immunogenicity of compositions and vaccine compositions against SARS-CoV-2. In embodiments, the immunogenic compositions and vaccine compositions target a SARS-CoV-2 virus, or a heterogeneous SARS-CoV-2 strain. In embodiments, the immunogenic composition or vaccine composition comprises a SARS-CoV-2 S glycoprotein or a nucleic acid (e.g., mRNA) that encodes a SARS-CoV-2 S glycoprotein. In embodiments, the immunogenic composition or vaccine composition comprises a viral vector that expresses a SARS-CoV-2 S glycoprotein. In embodiments, the immunogenic compositions or vaccine compositions comprise a SARS-CoV-2 S glycoprotein that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 1. In embodiments, the SARS-CoV-2 S glycoprotein comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 2. In embodiments, SARS-CoV-2 S glycoprotein comprises a sequence that is at least 90%, at least 91%, at least 92%, at least 93%, at least 94%, at least 95%, at least 96%, at least 97%, at least 98%, at least 99%, or 100% identical to SEQ ID NO: 3. The amino acid sequences of SEQ ID NOS: 1-3 are in the table below.

| SEQ ID | |
|---|---|
| NO: | Amino Acid Sequence of SARS-CoV-2 S Glycoprotein |
| 3 | QCVNLTTRTQLPPAYTNSFTRGVYYPDKVFRSSVLHSTQDLFLPFFSNVTWFHAIHVSGT |
| | NGTKRFDNPVLPFNDGVYFASTEKSNIIRGWIFGTTLDSKTQSLLIVNNATNVVIKVCEF |
| | QFCNDPFLGVYYHKNNKSWMESEFRVYSSANNCTFEYVSQPFLMDLEGKQGNFKNLREFV |
| | FKNIDGYFKIYSKHTPINLVRDLPQGFSALEPLVDLPIGINITRFQTLLALHRSYLTPGD |
| | SSSGWTAGAAAYYVGYLQPRTFLLKYNENGTITDAVDCALDPLSETKCTLKSFTVEKGIY |
| | QTSNFRVQPTESIVRFPNITNLCPFGEVFNATRFASVYAWNRKRISNCVADYSVLYNSAS |
| | FSTFKCYGVSPTKLNDLCFTNVYADSFVIRGDEVRQIAPGQTGKIADYNYKLPDDFTGCV |
| | IAWNSNNLDSKVGGNYNYLYRLFRKSNLKPFERDISTEIYQAGSTPCNGVEGFNCYFPLQ |
| | SYGFQPTNGVGYQPYRVVVLSFELLHAPATVCGPKKSTNLVKNKCVNFNFNGLTGTGVLT |
| | ESNKKFLPFQQFGRDIADTTDAVRDPQTLEILDITPCSFGGVSVITPGTNTSNQVAVLYQ |
| | DVNCTEVPVAIHADQLTPTWRVYSTGSNVFQTRAGCLIGAEHVNNSYECDIPIGAGICAS |
| | YQTQTNSPQQAQSVASQSIIAYTMSLGAENSVAYSNNSIAIPTNFTISVTTEILPVSMTK |
| | TSVDCTMYICGDSTECSNLLLQYGSFCTQLNRALTGIAVEQDKNTQEVFAQVKQIYKTPP |
| | IKDFGGFNFSQILPDPSKPSKRSFIEDLLFNKVTLADAGFIKQYGDCLGDIAARDLICAQ |
| | KFNGLTVLPPLLTDEMIAQYTSALLAGTITSGWTFGAGAALQIPFAMQMAYRFNGIGVTQ |
| | NVLYENQKLIANQFNSAIGKIQDSLSSTASALGKLQDVVNQNAQALNTLVKQLSSNFGAI |
| | SSVLNDILSRLDPPEAEVQIDRLITGRLQSLQTYVTQQLIRAAEIRASANLAATKMSECV |
| | LGQSKRVDFCGKGYHLMSFPQSAPHGVVFLHVTYVPAQEKNFTTAPAICHDGKAHFPREG |
| | VFVSNGTHWFVTQRNFYEPQIITTDNTFVSGNCDVVIGIVNNTVYDPLQPELDSFKEELD |
| | KYFKNHTSPDVDLGDISGINASVVNIQKEIDRLNEVAKNLNESLIDLQELGKYEQYIKWP |
| | WYIWLGFIAGLIAIVMVTIMLCCMTSCCSCLKGCCSCGSCCKFDEDDSEPVLKGVKLHYT |
| 1 | MFVFLVLLPLVSSQCVNLTTRTQLPPAYTNSFTRGVYYPDKVFRSSVLHSTQDLFLPFFS |
| | NVTWFHAIHVSGTNGTKRFDNPVLPFNDGVYFASTEKSNIIRGWIFGTTLDSKTQSLLIV |
| | NNATNVVIKVCEFQFCNDPFLGVYYHKNNKSWMESEFRVYSSANNCTFEYVSQPFLMDLE |
| | GKQGNFKNLREFVFKNIDGYFKIYSKHTPINLVRDLPQGFSALEPLVDLPIGINITRFQT |
| | LLALHRSYLTPGDSSSGWTAGAAAYYVGYLQPRTFLLKYNENGTITDAVDCALDPLSETK |
| | CTLKSFTVEKGIYQTSNFRVQPTESIVRFPNITNLCPFGEVFNATRFASVYAWNRKRISN |
| | CVADYSVLYNSASFSTFKCYGVSPTKLNDLCFTNVYADSFVIRGDEVRQIAPGQTGKIAD |
| | YNYKLPDDFTGCVIAWNSNNLDSKVGGNYNYLYRLFRKSNLKPFERDISTEIYQAGSTPC |
| | NGVEGFNCYFPLQSYGFQPTNGVGYQPYRVVVLSFELLHAPATVCGPKKSTNLVKNKCVN |
| | FNFNGLTGTGVLTESNKKFLPFQQFGRDIADTTDAVRDPQTLEILDITPCSFGGVSVITP |
| | GTNTSNQVAVLYQDVNCTEVPVAIHADQLTPTWRVYSTGSNVFQTRAGCLIGAEHVNNSY |
| | ECDIPIGAGICASYQTQTNSPRRARSVASQSIIAYTMSLGAENSVAYSNNSIAIPTNFTI |
| | SVTTEILPVSMTKTSVDCTMYICGDSTECSNLLLQYGSFCTQLNRALTGIAVEQDKNTQE |
| | VFAQVKQIYKTPPIKDFGGFNFSQILPDPSKPSKRSFIEDLLFNKVTLADAGFIKQYGDC |
| | LGDIAARDLICAQKFNGLTVLPPLLTDEMIAQYTSALLAGTITSGWTFGAGAALQIPFAM |
| | QMAYRFNGIGVTQNVLYENQKLIANQFNSAIGKIQDSLSSTASALGKLQDVVNQNAQALN |
| | TLVKQLSSNFGAISSVLNDILSRLDKVEAEVQIDRLITGRLQSLQTYVTQQLIRAAEIRA |
| | SANLAATKMSECVLGQSKRVDFCGKGYHLMSFPQSAPHGVVFLHVTYVPAQEKNFTTAPA |
| | ICHDGKAHFPREGVFVSNGTHWFVTQRNFYEPQIITTDNTFVSGNCDVVIGIVNNTVYDP |
| | LQPELDSFKEELDKYFKNHTSPDVDLGDISGINASVVNIQKEIDRLNEVAKNLNESLIDL |
| | QELGKYEQYIKWPWYIWLGFIAGLIAIVMVTIMLCCMTSCCSCLKGCCSCGSCCKFDEDD |
| | SEPVLKGVKLHYT |
| 2 | QCVNLTTRTQLPPAYTNSFTRGVYYPDKVFRSSVLHSTQDLFLPFFSNVTWFHAIHVSGT |
| | NGTKRFDNPVLPFNDGVYFASTEKSNIIRGWIFGTTLDSKTQSLLIVNNATNVVIKVCEF |
| | QFCNDPFLGVYYHKNNKSWMESEFRVYSSANNCTFEYVSQPFLMDLEGKQGNFKNLREFV |
| | FKNIDGYFKIYSKHTPINLVRDLPQGFSALEPLVDLPIGINITRFQTLLALHRSYLTPGD |
| | SSSGWTAGAAAYYVGYLQPRTFLLKYNENGTITDAVDCALDPLSETKCTLKSFTVEKGIY |
| | QTSNFRVQPTESIVRFPNITNLCPFGEVFNATRFASVYAWNRKRISNCVADYSVLYNSAS |
| | FSTFKCYGVSPTKLNDLCFTNVYADSFVIRGDEVRQIAPGQTGKIADYNYKLPDDFTGCV |
| | IAWNSNNLDSKVGGNYNYLYRLFRKSNLKPFERDISTEIYQAGSTPCNGVEGFNCYFPLQ |
| | SYGFQPTNGVGYQPYRVVVLSFELLHAPATVCGPKKSTNLVKNKCVNFNFNGLTGTGVLT |
| | ESNKKFLPFQQFGRDIADTTDAVRDPQTLEILDITPCSFGGVSVITPGTNTSNQVAVLYQ |
| | DVNCTEVPVAIHADQLTPTWRVYSTGSNVFQTRAGCLIGAEHVNNSYECDIPIGAGICAS |
| | YQTQTNSPRRARSVASQSIIAYTMSLGAENSVAYSNNSIAIPTNFTISVTTEILPVSMTK |
| | TSVDCTMYICGDSTECSNLLLQYGSFCTQLNRALTGIAVEQDKNTQEVFAQVKQIYKTPP |
| | IKDFGGFNFSQILPDPSKPSKRSFIEDLLFNKVTLADAGFIKQYGDCLGDIAARDLICAQ |
| | KFNGLTVLPPLLTDEMIAQYTSALLAGTITSGWTFGAGAALQIPFAMQMAYRFNGIGVTQ |
| | NVLYENQKLIANQFNSAIGKIQDSLSSTASALGKLQDVVNQNAQALNTLVKQLSSNFGAI |
| | SSVLNDILSRLDKVEAEVQIDRLITGRLQSLQTYVTQQLIRAAEIRASANLAATKMSECV |
| | LGQSKRVDFCGKGYHLMSFPQSAPHGVVFLHVTYVPAQEKNFTTAPAICHDGKAHFPREG |
| | VFVSNGTHWFVTQRNFYEPQIITTDNTFVSGNCDVVIGIVNNTVYDPLQPELDSFKEELD |
| | KYFKNHTSPDVDLGDISGINASVVNIQKEIDRLNEVAKNLNESLIDLQELGKYEQYIKWP |
| | WYIWLGFIAGLIAIVMVTIMLCCMTSCCSCLKGCCSCGSCCKEDEDDSEPVLKGVKLHYT |

In embodiments, the immunogenic compositions or vaccine compositions comprise an adjuvant. Exemplary adjuvants are described below.

### Aluminum-Based Adjuvants

In embodiments, the adjuvant may be alum (e.g. AlPO₄ or Al(OH)₃). Typically, the nanoparticle is substantially bound to the alum. For example, the nanoparticle may be at least 80% bound, at least 85% bound, at least 90% bound or at least 95% bound to the alum. Often, the nanoparticle is 92% to 97% bound to the alum in a composition. The amount of alum is present per dose is typically in a range between about 400 μg to about 1250 μg. For example, the alum may be present in a per dose amount of about 300 μg to about 900 μg, about 400 μg to about 800 μg, about 500 μg to about 700 μg, about 400 μg to about 600 μg, or about 400 μg to about 500 μg. Typically, the alum is present at about 400 μg for a dose of 120 μg of the protein nanoparticle.

### Saponin Adjuvants

Adjuvants containing saponin may also be combined with the immunogens disclosed herein. Saponins are glycosides derived from the bark of the Quillaja saponaria Molina tree. Typically, saponin is prepared using a multi-step purification process resulting in multiple fractions. As used, herein, the term “a saponin fraction from Quillaja saponaria Molina” is used generically to describe a semi-purified or defined saponin fraction of Quillaja saponaria or a substantially pure fraction thereof.

### Saponin Fractions

Several approaches for producing saponin fractions are suitable. Fractions A, B, and C are described in U.S. Pat. No. 6,352,697 and may be prepared as follows. A lipophilic fraction from Quil A, a crude aqueous Quillaja saponaria Molina extract, is separated by chromatography and eluted with 70% acetonitrile in water to recover the lipophilic fraction. This lipophilic fraction is then separated by semi-preparative HPLC with elution using a gradient of from 25% to 60% acetonitrile in acidic water. The fraction referred to herein as “Fraction A” or “QH-A” is, or corresponds to, the fraction, which is eluted at approximately 39% acetonitrile. The fraction referred to herein as “Fraction B” or “QH-B” is, or corresponds to, the fraction, which is eluted at approximately 47% acetonitrile. The fraction referred to herein as “Fraction C” or “QH-C” is, or corresponds to, the fraction, which is eluted at approximately 49% acetonitrile. Additional information regarding purification of Fractions is found in U.S. Pat. No. 5,057,540. When prepared as described herein, Fractions A, B and C of Quillaja saponaria Molina each represent groups or families of chemically closely related molecules with definable properties. The chromatographic conditions under which they are obtained are such that the batch-to-batch reproducibility in terms of elution profile and biological activity is highly consistent.

Other saponin fractions have been described. Fractions B3, B4 and B4b are described in EP 0436620. Fractions QA1-QA22 are described EP03632279 B2, Q-VAC (Nor-Feed, AS Denmark), Quillaja saponaria Molina Spikoside (lsconova AB, Ultunaallén 2B, 756 51 Uppsala, Sweden). Fractions QA-1, QA-2, QA-3, QA-4, QA-5, QA-6, QA-7, QA-8, QA-9, QA-10, QA-11, QA-12, QA-13, QA-14, QA-15, QA-16, QA-17, QA-18, QA-19, QA-20, QA-21, and QA-22 of EP 0 3632 279 B2, especially QA-7, QA-17, QA-18, and QA-21 may be used. They are obtained as described in EP 0 3632 279 B2, especially at page 6 and in Example 1 on page 8 and 9.

The saponin fractions described herein and used for forming adjuvants are often substantially pure fractions; that is, the fractions are substantially free of the presence of contamination from other materials. In particular aspects, a substantially pure saponin fraction may contain up to 40% by weight, up to 30% by weight, up to 25% by weight, up to 20% by weight, up to 15% by weight, up to 10% by weight, up to 7% by weight, up to 5% by weight, up to 2% by weight, up to 1% by weight, up to 0.5% by weight, or up to 0.1% by weight of other compounds such as other saponins or other adjuvant materials.

### ISCOM Structures

Saponin fractions may be administered in the form of a cage-like particle referred to as an ISCOM (Immune Stimulating COMplex). ISCOMs may be prepared as described in EP0109942B1, EP0242380B1 and EP0180546 B1. In particular embodiments a transport and/or a passenger antigen may be used, as described in EP 9600647-3 (PCT/SE97/00289).

### Matrix Adjuvants

In embodiments, the ISCOM is an ISCOM matrix complex. An ISCOM matrix complex comprises at least one saponin fraction and a lipid. The lipid is at least a sterol, such as cholesterol. In particular aspects, the ISCOM matrix complex also contains a phospholipid. The ISCOM matrix complexes may also contain one or more other immunomodulatory (adjuvant-active) substances, not necessarily a glycoside, and may be produced as described in EP0436620B1, which is incorporated by reference in its entirety herein.

In other aspects, the ISCOM is an ISCOM complex. An ISCOM complex contains at least one saponin, at least one lipid, and at least one kind of antigen or epitope. The ISCOM complex contains antigen associated by detergent treatment such that that a portion of the antigen integrates into the particle. In contrast, ISCOM matrix is formulated as an admixture with antigen and the association between ISCOM matrix particles and antigen is mediated by electrostatic and/or hydrophobic interactions.

According to one embodiment, the saponin fraction integrated into an ISCOM matrix complex or an ISCOM complex, or at least one additional adjuvant, which also is integrated into the ISCOM or ISCOM matrix complex or mixed therewith, is selected from fraction A, fraction B, or fraction C of Quillaja saponaria, a semipurified preparation of Quillaja saponaria, a purified preparation of Quillaja saponaria, or any purified sub-fraction e.g., QA 1-21.

In particular aspects, each ISCOM particle may contain at least two saponin fractions. Any combinations of weight % of different saponin fractions may be used. Any combination of weight % of any two fractions may be used. For example, the particle may contain any weight % of fraction A and any weight % of another saponin fraction, such as a crude saponin fraction or fraction C, respectively. Accordingly, in particular aspects, each ISCOM matrix particle or each ISCOM complex particle may contain from 0.1 to 99.9 by weight, 5 to 95% by weight, 10 to 90% by weight 15 to 85% by weight, 20 to 80% by weight, 25 to 75% by weight, 30 to 70% by weight, 35 to 65% by weight, 40 to 60% by weight, 45 to 55% by weight, 40 to 60% by weight, or 50% by weight of one saponin fraction, e.g. fraction A and the rest up to 100% in each case of another saponin e.g. any crude fraction or any other faction e.g. fraction C. The weight is calculated as the total weight of the saponin fractions. Examples of ISCOM matrix complex and ISCOM complex adjuvants are disclosed in U.S. Published Application No. 2013/0129770, which is incorporated by reference in its entirety herein.

In particular embodiments, the ISCOM matrix or ISCOM complex comprises from 5-99% by weight of one fraction, e.g. fraction A and the rest up to 100% of weight of another fraction e.g. a crude saponin fraction or fraction C. The weight is calculated as the total weight of the saponin fractions.

In another embodiment, the ISCOM matrix or ISCOM complex comprises from 40% to 99% by weight of one fraction, e.g. fraction A and from 1% to 60% by weight of another fraction, e.g. a crude saponin fraction or fraction C. The weight is calculated as the total weight of the saponin fractions.

In yet another embodiment, the ISCOM matrix or ISCOM complex comprises from 70% to 95% by weight of one fraction e.g., fraction A, and from 30% to 5% by weight of another fraction, e.g., a crude saponin fraction, or fraction C. The weight is calculated as the total weight of the saponin fractions. In other embodiments, the saponin fraction from Quillaja saponaria Molina is selected from any one of QA 1-21.

In addition to particles containing mixtures of saponin fractions, ISCOM matrix particles and ISCOM complex particles may each be formed using only one saponin fraction. Compositions disclosed herein may contain multiple particles wherein each particle contains only one saponin fraction. That is, certain compositions may contain one or more different types of ISCOM-matrix complexes particles and/or one or more different types of ISCOM complexes particles, where each individual particle contains one saponin fraction from Quillaja saponaria Molina, wherein the saponin fraction in one complex is different from the saponin fraction in the other complex particles.

In particular aspects, one type of saponin fraction or a crude saponin fraction may be integrated into one ISCOM matrix complex or particle and another type of substantially pure saponin fraction, or a crude saponin fraction, may be integrated into another ISCOM matrix complex or particle. A composition or vaccine may comprise at least two types of complexes or particles each type having one type of saponins integrated into physically different particles.

In the compositions, mixtures of ISCOM matrix complex particles and/or ISCOM complex particles may be used in which one saponin fraction Quillaja saponaria Molina and another saponin fraction Quillaja saponaria Molina are separately incorporated into different ISCOM matrix complex particles and/or ISCOM complex particles.

The ISCOM matrix or ISCOM complex particles, which each have one saponin fraction, may be present in composition at any combination of weight %. In particular aspects, a composition may contain 0.1% to 99.9% by weight, 5% to 95% by weight, 10% to 90% by weight, 15% to 85% by weight, 20% to 80% by weight, 25% to 75% by weight, 30% to 70% by weight, 35% to 65% by weight, 40% to 60% by weight, 45% to 55% by weight, 40 to 60% by weight, or 50% by weight, of an ISCOM matrix or complex containing a first saponin fraction with the remaining portion made up by an ISCOM matrix or complex containing a different saponin fraction. In aspects, the remaining portion is one or more ISCOM matrix or complexes where each matrix or complex particle contains only one saponin fraction. In other aspects, the ISCOM matrix or complex particles may contain more than one saponin fraction.

In particular compositions, the only saponin fraction in a first ISCOM matrix or ISCOM complex particle is Fraction A and the only saponin fraction in a second ISCOM matrix or ISCOM complex particle is Fraction C.

In embodiments, the Fraction A of Quillaja saponaria Molina accounts for at least about 80%, 81%, 82%, 83%, 84%, 85%, 86%, 87%, 88%, 89%, 90%, 91%, 92%, 93%, 94%, 95%, 96%, 97%, 98%, or 99% by weight, and fraction C of Quillaja saponaria Molina accounts for the remainder, respectively, of the sum of the weights of fraction A of Quillaja saponaria Molina and fraction C of Quillaja saponaria Molina in the adjuvant.

Preferred compositions comprise a first ISCOM matrix containing Fraction A and a second ISCOM matrix containing Fraction C, wherein the Fraction A ISCOM matrix constitutes about 70% per weight of the total saponin adjuvant, and the Fraction C ISCOM matrix constitutes about 30% per weight of the total saponin adjuvant. In another preferred composition, the Fraction A ISCOM matrix constitutes about 85% per weight of the total saponin adjuvant, and the Fraction C ISCOM matrix constitutes about 15% per weight of the total saponin adjuvant. In another preferred composition, the Fraction A ISCOM matrix constitutes about 92% per weight of the total saponin adjuvant, and the Fraction C ISCOM matrix constitutes about 8% per weight of the total saponin adjuvant. Thus, in certain compositions, the Fraction A ISCOM matrix is present in a range of about 70% to about 85%, and Fraction C ISCOM matrix is present in a range of about 15% to about 30%, of the total weight amount of saponin adjuvant in the composition. In certain compositions, the Fraction A ISCOM matrix is present in a range of about 70% to about 92%, and Fraction C ISCOM matrix is present in a range of about 8% to about 30%, of the total weight amount of saponin adjuvant in the composition. In embodiments, the Fraction A ISCOM matrix accounts for 50-96% by weight and Fraction C ISCOM matrix accounts for the remainder, respectively, of the sums of the weights of Fraction A ISCOM matrix and Fraction C ISCOM in the adjuvant. In a particularly preferred composition, referred to herein as MATRIX-M™, the Fraction A ISCOM matrix is present at about 85% and Fraction C ISCOM matrix is present at about 15% of the total weight amount of saponin adjuvant in the composition. MATRIX-M™ may be referred to interchangeably as Matrix-M1.

Exemplary QS-7 and QS-21 fractions, their production and their use is described in U.S. Pat. Nos. 5,057,540; 6,231,859; 6,352,697; 6,524,584; 6,846,489; 7,776,343, and 8,173,141, which are incorporated by reference herein.

In embodiments, other adjuvants may be used in addition or as an alternative. The inclusion of any adjuvant described in Vogel et al., “A Compendium of Vaccine Adjuvants and Excipients (2nd Edition),” herein incorporated by reference in its entirety for all purposes, is envisioned within the scope of this disclosure. Other adjuvants include complete Freund's adjuvant (a non-specific stimulator of the immune response containing killed Mycobacterium tuberculosis), incomplete Freund's adjuvants and aluminum hydroxide adjuvant. Other adjuvants comprise GMCSP, BCG, MDP compounds, such as thur-MDP and nor-MDP, CGP (MTP-PE), lipid A, and monophosphoryl lipid A (MPL), MF-59, RIBI, which contains three components extracted from bacteria, MPL, trehalose dimycolate (TDM) and cell wall skeleton (CWS) in a 2% squalene/TWEEN® polysorbate 80 emulsion. In embodiments, the adjuvant may be a Pauci lamellar lipid vesicle; for example, NOVASOMES®. NOVASOMES® are Pauci lamellar nonphospholipid vesicles ranging from about 100 nm to about 500 nm. They comprise BRIJ® alcohol ethoxylate 72, cholesterol, oleic acid and squalene. NOVASOMES® have been shown to be an effective adjuvant (see, U.S. Pat. Nos. 5,629,021, 6,387,373, and 4,911,928.

### Example 1. SARS-CoV-2 Receptor (ACE2) Inhibition Assay: a Rapid High Throughput Assay Useful for Vaccine Immunogenicity Evaluation

We have discovered a rapid high throughput option to evaluate vaccine immunogenicity. The assay is a BSL2 based assay, which is cost effective and rapid. The assay allows for an evaluation of vaccine immunogenicity against emerging SARS-CoV-2 variants. This assay may replace live virus neutralization assays.

Introduction: Emerging severe acute respiratory syndrome coronavirus 2 (SARS-CoV-2) virus variants escape protective immunity generated by currently available vaccines, underscoring the need for better clinical immunogenicity biomarkers.

Method: One novel biomarker is the inhibition of the interaction between hACE2 and a SARS-CoV-2 S glycoprotein. To measure this biomarker, we developed and validated an enzyme-linked immunosorbent assay (ELISA) assay to measure inhibition between a SARS-CoV-2 S glycoprotein and hACE2. The assay was validated for precision, specificity, and linearity.

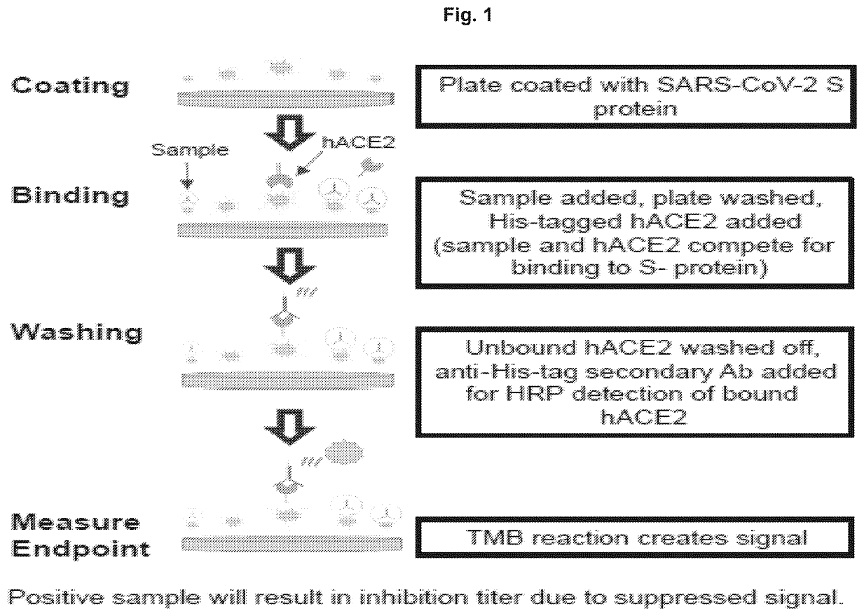

ELISA Assay: A 96-well format, enzyme-linked immunosorbent assay-based assay was developed to assess inhibition of binding of prototype/Wuhan strain or variant (Delta, Omicron BA.1/BA.5) SARS-CoV-2 S-protein (full-length rS protein) to the receptor hACE2 by human sera from clinical trials of the NVX-CoV2373 vaccine. The following SARS-CoV-2 S glycoproteins were evaluated: a SARS-CoV-2 S glycoprotein of SEQ ID NO: 2 (prototype, Wuhan); a SARS-CoV-2 S glycoprotein from the SARS-CoV-2 Delta Strain; a SARS-CoV-2 S glycoprotein from an Omicron BA.1 strain; and a SARS-CoV-2 S glycoprotein from an Omicron BA.5 strain. FIG. 1 shows a schematic of the assay. The 96-well assay plates (Thermo Fisher Scientific, Waltham, MA, USA) were coated with SARS-CoV-2 rS-protein (produced at the Novavax, Inc. Gaithersburg, MD, USA) using a standard plate-coating method overnight at 2-8° C. This was followed by washing with phosphate-buffered saline with Tween 20 (PBST) and blocking with blocking buffer (Thermo Fisher Scientific) for 1 hour. Diluted serum samples were then added to the plate, followed by washing with PBST and the addition of polyhistidine-tagged hACE2. After unbound hACE2 was washed away with PBST, bound hACE2 was detected by incubating the plate with anti-polyhistidine-tagged/horseradish peroxidase secondary antibodies at room temperature (RT) for 1 hour, then washing with PBST, followed by incubation with 3,3′5,5′-tetramethylbenzidine substrate (Thermo Fisher Scientific) for 30 minutes. Quantitation of positive 3,3′5,5′-tetramethylbenzidine reaction signal indicates the level of inhibition of hACE2 binding by serum components, as the amount of bound hACE2 giving the signal is inversely proportional to the amount of hACE2-binding inhibitors in the serum. The 50% inhibitory titer was calculated using a 4-parameter logistical fit and was compared with hACE2 controls.

Samples: The following samples were evaluated: (i) human serum samples collected in 2018 before the COVID-19 pandemic (n=13); serum samples collected during the COVID-19 pandemic (n=26); serum samples from patients who recovered from SARS-CoV-2 infection (convalescent serum, n=24), or who were vaccinated with the NVX-CoV2373 vaccine; serum samples from individuals vaccinated with an influenza vaccine; and quality control (QC) samples containing pooled human serum samples that was either positive or negative in the hACE2 binding inhibition assay. Negative controls were pre-pandemic sera negative for hACE2 binding inhibition in the assay. QC samples were tested in duplicate on the first plate of each run.

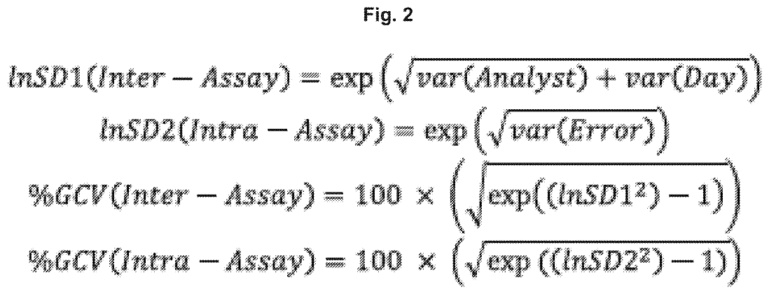

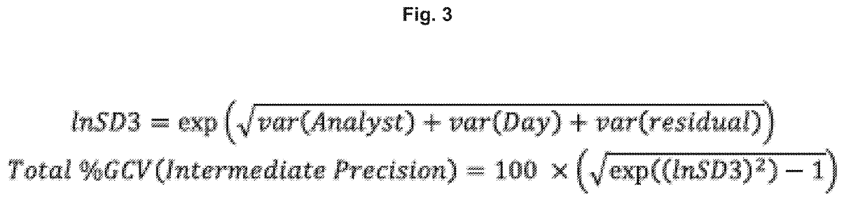

Validation Assay—Precision: Twenty samples were tested twice in an assay run (total of 6 runs by 2 analysts on 3 days), and each sample was tested in duplicate, with geometric mean titer (GMT) of duplicate values considered the inhibition titer. Precision was then estimated by calculating percent geometric coefficient of variation (% GCV) based on variance component analysis using sample as a fixed effect and analyst and day as the random effects. Target precision was such that at least 80% of samples have a % GCV≤20%, and % GCV≤25% for samples at lower limit of quantitation (LLoQ). % GCV was calculated using the formulas in FIG. 2 based on the variance component analysis using sample as a fixed effect and analyst and day as the random effects. The formula for total % GCV is found in FIG. 3. Target precision was set at at least 80% of samples having % GCV≤20%, and % GCV≤25% for samples at the lower limit of quantitation (LLOQ).

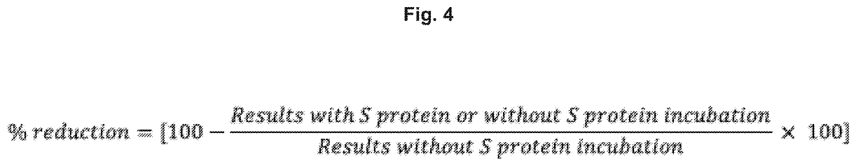

Validation Assay—Specificity: hACE2 binding inhibition-positive serum samples were incubated with rS protein for about 1 hour at RT before testing (5 samples). Controls used to set baseline were the same samples incubated with assay buffer only The irrelevant nonspecific protein group used the same samples but incubated with respiratory syncytial virus (RSV) F protein or Ebola glycoprotein (GP), produced using same recombinant protein platform as for rS protein. Samples were then tested in the assay and inhibition titers were compared, with % reduction calculated as in FIG. 4.

Validation Assay—Selectivity: Nineteen samples collected before the COVID-19 pandemic (assumed to be negative for SARS-CoV-2 antibodies) and expected to be below the LLoQ were tested for hACE2 binding inhibition titers. Some samples were also tested for influenza hemagglutination inhibition (HAI) titers to assess whether varying levels of HAI titers interfered with detection of hACE2 binding inhibition titers

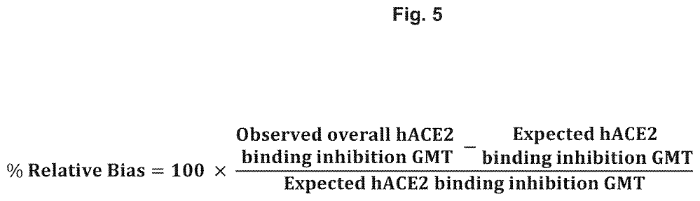

Validation Assay—Linearity: Two hACE2 binding inhibition-positive samples were tested in the assay undiluted or in a 1:2 dilution series (5 assays by different analysts), precision and accuracy of titer were calculated at each dilution point, and linear regression was conducted for observed versus expected GMT. Expected titer at each dilution was calculated from the overall GMT from all runs of the least diluted sample divided by the dilution factor, and observed GMT was the overall GMT from all runs. The % relative bias at each dilution point was calculated as in FIG. 5.

Validation Assay—Sensitivity: The lowest (LLoQ) titer values that were accurately and precisely determined were assessed for the 2 samples in the linearity analysis. The LLoQ for the assay was set at 10 based on prior data accumulated with convalescent sera from COVID-19 cases; data developed here in both precision and dilutional linearity experiments confirmed the LLoQ based on acceptable GCVs obtained for samples and dilutions with results <15.

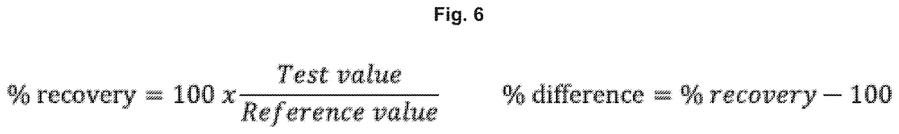

Validation Assay—Incubation Time Robustness: The assay was conducted using upper and lower incubation time limits for each step, then results were compared with the 6 runs done for the precision analysis (reference condition). The following time conditions (lower/upper limit) were used: plate coating (14/72 hours, reference 18-20 hours), plate blocking (60/90 minutes, reference 60 minutes), sample incubation (55/62 or 65 minutes, reference 60 minutes), hACE2 incubation (55/65 minutes, reference 60 minutes), secondary antibody (55/65 minutes, reference 60 minutes), TMB incubation (25/35 minutes, reference 30 minutes). The target was such that ≥80% of samples should have values within ±20% of the reference (80-120% of reference values). % recovery and % difference were calculated as in FIG. 6.

Validation Assay—Sample Stability: Sample stability—samples were stored at various temperatures (6 hours or 24 hours at RT [n=16], 7 days or 14 days in 2-8° C. [n=15], 9 months or 24 months at −80±10° C. [n=16]), then tested in the assay and the results were compared to freshly thawed samples (RT/fridge) or the original precision results (freezer). Samples at RT were thawed and then put in a 24±2° C. incubator for 6 hours or 24 hours. The samples stored at −80° C. were aliquots of the original samples from the precision assay. Samples were also tested after undergoing 7 or 8 freeze/thaw cycles (1 hour at RT followed by refreezing), and results were compared with aliquots of the same samples that underwent only 1 freeze/thaw cycle. % recovery was calculated for all samples as in FIG. 6. Only convalescent serum was used, as clinical trial samples were not available at the time of testing.

Validation Assay—Matrix Effects: For hemolysis analyses, hemolyzed human serum was spiked into 5 samples and a negative control sample to create 50% hemolyzed or 25% hemolyzed samples (representing severe hemolysis). The samples were then tested in the assay, and percent recovery was calculated as in FIG. 6 and compared with normal samples without hemolysis. For lipemia analyses, serum with high levels of triglycerides was spiked into 6 samples to create samples with a final triglyceride concentration of 500 or 250 mg/dL (normal level, <150 mg/dL). The samples were then tested in the assay, and percent recovery was calculated as shown in FIG. 6 and compared with normal samples without lipemia.

Variant Assays: The assay validation method and protocol for the variants (Delta, Omicron BA.1/BA.5) followed a similar experimental, qualification, and assay validation plan as for proto-type/Wuhan strain, but the S protein coated onto the plate was replaced with proteins reflecting the respective variant sequences. Assay precision, dilution linearity, assay specificity, selectivity, LLoQ/upper level of quantification (ULoQ), and assay robustness (coating time) were assessed for each variant. Matrix interference, assay robustness (incubation times), and sample stability were assessed as part of the validation process for the original prototype/Wuhan strain assay.

Correlation Analyses: For each sample, detection of anti-rS IgG antibodies and microneutralization antibodies was performed using methods previously described, then results were compared with hACE2 binding inhibition assay titers, and linear regression analysis was performed using GraphPad Prism software (San Diego, CA; Version 9.3.1). For some serum samples, hACE2 binding inhibition assay results for prototype and Omicron BA.1 were observed at limit of detection (LOD) levels, most probably because of low levels of hACE2 binding inhibition. To avoid the effect of these samples on the correlations, correlation analysis was performed with and without including the hACE2 binding inhibition results at LOD. As there was no significant difference in the strength of correlations, plots of hACE2 binding inhibition versus anti-rS IgG levels for prototype and Omicron BA.1 excluding the data points at LOD are shown in the main text.

Results: Interassay and intra-assay precision was <2000 GCV for all 20 convalescent serum samples available at the time of assay development. Total assay precision for 90% of samples (18/20) was <20% GCV, meeting acceptance criteria (Table 1). Later, 21 serum samples from vaccinated individuals also showed <200% GCV, and represented a wide range of titers (data not shown).

TABLE 1

| | | hACE2 | | | |
|---|---|---|---|---|---|
| | | Binding | | | |
| | | Inhibition | Inter- | Intra- | |
| | Sample | Titer | assay % | assay % | Total % |
| Sample | ID | (GMT) | GCV | GCV | GCV |
| 1 | HQC | 158.5 | 8.2 | 6.5 | 10.5 |
| 2 | LQC | 37.5 | 8.3 | 7.4 | 11.1 |
| 3 | MQC | 66.6 | 8.0 | 6.9 | 10.5 |
| 4 | NC | 5.0 | 0.0 | 0.0 | 0.0 |
| 5 | Serum #3 | 81.1 | 15.3 | 16.1 | 22.3 |
| 6 | Serum #1 | 91.3 | 18.8 | 7.3 | 20.2 |
| 7 | Serum #2 | 62.4 | 7.4 | 8.5 | 11.3 |
| 8 | Serum #12 | 11.0 | 13.0 | 12.7 | 18.2 |
| 9 | Serum #13 | 115.5 | 11.4 | 6.2 | 13.0 |
| 10 | Serum #14 | 22.3 | 6.2 | 7.3 | 9.6 |
| 11 | Serum #15 | 170.3 | 7.6 | 9.2 | 11.9 |
| 12 | Serum #16 | 54.7 | 11.5 | 11.3 | 16.2 |
| 13 | Serum #17 | 93.7 | 10.2 | 6.7 | 12.3 |
| 14 | Serum #18 | 22.4 | 8.2 | 8.8 | 12.1 |
| 15 | Serum #19 | 61.6 | 10.3 | 7.6 | 12.8 |
| 16 | Serum #20 | 18.0 | 4.3 | 10.2 | 11.1 |
| 17 | Serum #21 | 26.9 | 12.3 | 8.3 | 14.9 |
| 18 | Serum #22 | 27.9 | 8.1 | 7.4 | 11.0 |
| 19 | Serum #23 | 62.3 | 7.3 | 8.6 | 11.3 |
| 20 | Serum #24 | 13.6 | 12.2 | 12.6 | 17.6 |

% GCV—percent geometric coefficient of variation; GMT—geometric mean titer; hACE2—human angiotensin-converting enzyme 2; HQC—high quality control; ID—identification; LQC—low quality control; MQC—mid quality control; NC—negative control.

Assay specificity met the acceptance criteria. All tested samples showed at least 74.10 reduction in hACE2 binding inhibition titers after preincubation of sera with SARS-CoV-2 rS-protein (≥50% reduction needed for acceptance). The samples also showed less than 200% change in inhibitory titer for irrelevant protein, when incubated with RSV F protein or Ebola GP (Table 2A). Assay selectivity met the acceptance criteria, as all samples collected before the pandemic showed negative (<LLoQ) results for hACE2 binding inhibition. Paired pre/post (day 0/day 21) samples from participants showing strong responses to influenza immunization showed that detection was not affected by large vaccine-induced changes in HAI titers (Table 2B).

TABLE 2A

| | | Incubated with | | Incubated | | Incubated | |
|---|---|---|---|---|---|---|---|
| | | SARS-CoV-2 S | | with RSV F | | with Ebola | |
| | | Glycoprotein | | Glycoprotein | | Glycoprotein | |
| | Assay | | % | | % | | % |
| Sample | Buffer | | Reduc- | | Reduc- | | Reduc- |
| ID | GMT | GMT | tion | GMT | tion | GMT | tion |
| Serum | 175.5 | 31.4 | 82.1 | 183.1 | −4.3 | 181.6 | −3.5 |
| #45 | | | | | | | |
| Serum | 85.8 | 21.1 | 75.4 | 79.3 | 7.6 | 92.6 | −7.8 |
| #17 | | | | | | | |
| Serum | 102.1 | 26.4 | 74.1 | 99.8 | 2.3 | 111.4 | −9.0 |
| #13 | | | | | | | |
| Serum | 24.7 | 5 | 79.7 | 26.8 | −8.5 | 26.5 | −7.5 |
| #22 | | | | | | | |
| Serum | 58.8 | 13.1 | 77.6 | 63.3 | −7.7 | 56.5 | 3.9 |
| #16 | | | | | | | |

TABLE 2B

| | | hACE2 | Anti-A/ | | Anti-A/ | |
|---|---|---|---|---|---|---|
| | | Binding | Singapore^{a} | | Michigan^{b} | |
| Sample ID | Sample # | Titer | HAI GMT | | HAI GMT | |
| Serum #39^{c} | 1 | <10 | 20 | (Day 0) | 40 | (day 0) |
| Serum #40^{c} | 2 | <10 | 2560 | (Day 21) | 2560 | (day 21) |
| Serum #41^{c} | 3 | <10 | 20 | (Day 0) | 40 | (day 0) |
| Serum #42^{c} | 4 | <10 | 2560 | (Day 21) | 40 | (day 21) |
| Serum #43^{c} | 5 | <10 | 40 | (Day 0) | 40 | (Day 0) |
| Serum #44^{c} | 6 | <10 | 2560 | (Day 21) | 2560 | (Day 21) |

| | | | | |
|---|---|---|---|---|
| Serum #25 | 7 | <10 | 640 | 151 |
| Serum #26 | 8 | <10 | 523 | 302 |
| Serum #27 | 9 | <10 | 95 | 80 |
| Serum #28 | 10 | <10 | N/A | 142 |
| Serum #29 | 11 | <10 | N/A | 36 |
| Serum #30 | 12 | <10 | N/A | 170 |
| Serum #31 | 13 | <10 | N/A | 640 |
| Serum #32 | 14 | <10 | N/A | N/A |
| Serum #33 | 15 | <10 | N/A | 60 |
| Serum #34 | 16 | <10 | N/A | N/A |
| Serum #35 | 17 | <10 | N/A | 285 |
| Serum #36 | 18 | <10 | N/A | 151 |
| Serum #37 | 19 | <10 | N/A | N/A |

^{a}influenza virus A/Singapore/INFIMH-16-0019/2016
^{b}influenza virus A/Michigan/45/2015
^{c}These samples are from 3 pairs of samples from 3 participants in the influenza vaccine trial

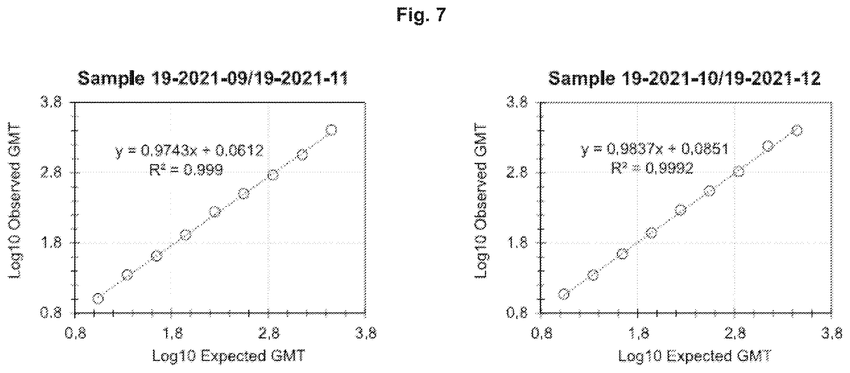

Linearity of the assay was successfully demonstrated, with R² values of 0.999 for both of the 2 individual samples examined (FIG. 7). LLoQ was assigned a titer of 10, based on the lowest titer values that were accurately and precisely detected for the 2 samples (expected titer of 8.3 or of 13.9). ULoQ was determined to be at least 2540.1 based on the highest hACE2 binding inhibition titers from the linearity analysis and based on clinical samples available at the time of validation of this assay. With the availability of additional samples from clinical trials, the accumulation of sera with titers greater the current ULoQ is expected. At regular time intervals, the precision and dilutional linearity of the assay for any such sera will be re-examined, and the ULoQ updated. Table 3A shows the relevant parameters associated with the charts in FIG. 2.

TABLE 3A

| Sample | Parameter | Estimate | 95% LCL | 95% UCL |
|---|---|---|---|---|
| 19-2021-09/ | Slope | 0.974 | 0.947 | 1.001 |
| 19-2021-11 | Intercept | 0.061 | −0.002 | 0.124 |
| | Residual | 0.027 | N/A | |
| | Variability | (6.3%) | | |
| | (% GSD) | | | |
| | R² | 0.999 | N/A | |
| 19-2020-10/ | Slope | 0.984 | 0.959 | 1.009 |
| 19-2020-12 | Intercept | 0.085 | 0.027 | 0.143 |
| | Residual | 0.025 | N/A | |
| | Variability | (5.8%) | | |
| | (% GSD) | | | |

| | | |
|---|---|---|
| R² | 0.999 | N/A |

There was minimal impact on the assay by the presence of free hemoglobin (Table 3B3) or a lipemic matrix. (Table 3C).

TABLE 3B
Heme Matrix Effects

| | Control | 50% Hemolyzed | | 25% Hemolyzed | |
|---|---|---|---|---|---|
| | Inhibition | Inhibition | | Inhibition | |
| Sample | GMT | GMT | Recovery | GMT | Recovery |
| Serum #62 | 72.1 | 63.2 | 87.7 | 54.7 | 75.9 |
| Serum #63 | 16.2 | 15.7 | 96.9 | 16.7 | 103.1 |
| Serum #64 | 106.1 | 118 | 111.2 | 136.4 | 128.6 |
| Serum #65 | 142.6 | 133.8 | 93.8 | 118.3 | 83 |
| Serum #66 | 42.4 | 42.6 | 100.5 | 41.5 | 97.9 |
| Serum #67 | <10 | <10 | N/A | <10 | N/A |

TABLE 3C
Lipid Matrix Effects

| | | 5 mg/mL | | 2.5 mg/mL | |
|---|---|---|---|---|---|
| | Control | triglycerides | | triglycerides | |
| | Inhibition | Inhibition | | Inhibition | |
| Sample | GMT | GMT | Recovery | GMT | Recovery |
| Serum #62 | 72.1 | 61.5 | 85.3 | 71.1 | 98.6 |
| Serum #63 | 16.2 | 21.7 | 134 | 20.2 | 124.7 |
| Serum #64 | 106.1 | 125.2 | 118 | 108.8 | 102.5 |
| Serum #65 | 142.6 | 118.6 | 83.2 | 111.2 | 78 |
| Serum #66 | 42.4 | 44.1 | 104 | 42 | 99.1 |
| Serum #67 | <10 | <10 | N/A | <10 | N/A |

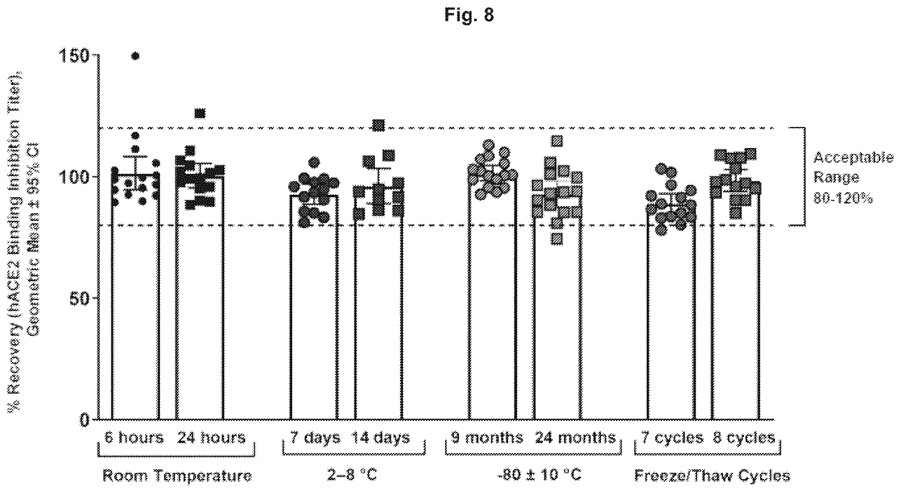

Stability of samples was tested at room temperature, refrigeration (2-8° C.), and freezing (−80° C.), and the samples were stable up to 8 freeze-thaw cycles and up to 24 months in freezer storage (FIG. 8).

Assay robustness for incubation time was demonstrated for the prototype strain, as 95% of samples at the lower limit of incubation time and 85% of samples at the upper limit of incubation time had hACE2 binding inhibition GMT between 80-120% of the reference condition values. (Tables 4+5)

TABLE 4
Assay Robustness-lower incubation time results

| | | Reference | Lower Incubation | | % | % |
|---|---|---|---|---|---|---|
| Sample | Sample | Condition | Time Limit | | Recov- | Differ- |
| # | ID | Titer | Titer^{a} | GMT | ery | ence |
| 1 | HQC | 158.5 | 148 | 141.6 | 89.3 | −10.7 |
| | | | 135.4 | | | |
| 2 | MQC | 66.6 | 61.4 | 60.9 | 60.9 | 91.5 |
| | | | 60.5 | | | |
| 3 | LQC | 37.6 | 35.8 | 35.6 | 35.6 | 94.7 |
| | | | 35.4 | | | |
| 4 | NC | 5 | 5 | 5 | 5 | 100 |
| | | | 5 | | | |
| 5 | Serum #12 | 11 | 8.7 | 8.5 | 8.5 | 77.7 |
| | | | 8.4 | | | |
| 6 | Serum #13 | 115.5 | 101.5 | 102.7 | 102.7 | 88.9 |
| | | | 103.9 | | | |
| 7 | Serum #14 | 22.3 | 19.5 | 19 | 19 | 85.4 |
| | | | 18.6 | | | |
| 8 | Serum #15 | 170.3 | 149.3 | 157.5 | 157.5 | 92.5 |
| | | | 166.2 | | | |
| 9 | Serum #16 | 54.7 | 48.6 | 47.4 | 47.4 | 86.6 |
| | | | 46.2 | | | |
| 10 | Serum #17 | 93.7 | 76 | 76.7 | 76.7 | 81.9 |
| | | | 77.4 | | | |
| 11 | Serum #18 | 22.4 | 21.8 | 21 | 21 | 93.9 |
| | | | 20.3 | | | |
| 12 | Serum #19 | 61.6 | 50.3 | 51.3 | 51.3 | 83.3 |
| | | | 52.4 | | | |
| 13 | Serum #20 | 18 | 19.1 | 17.7 | 17.7 | 98.3 |
| | | | 16.4 | | | |
| 14 | Serum #21 | 26.9 | 26.2 | 26.3 | 26.3 | 98 |
| | | | 26.5 | | | |
| 15 | Serum #22 | 27.9 | 26.1 | 26.3 | 26.3 | 94.4 |
| | | | 26.6 | | | |
| 16 | Serum #23 | 62.3 | 52.3 | 55.1 | 55.1 | 88.4 |
| | | | 58 | | | |
| 17 | Serum #24 | 13.6 | 14.9 | 14 | 14 | 103.1 |
| | | | 13.2 | | | |
| 18 | Serum #1 | 91.4 | 91.5 | 88.4 | 88.4 | 96.8 |
| | | | 85.5 | | | |
| 19 | Serum #2 | 62.4 | 54.9 | 58.5 | 58.5 | 93.7 |
| | | | 62.3 | | | |
| 20 | Serum #3 | 81.1 | 88.3 | 90.2 | 90.2 | 111.3 |
| | | | 92.2 | | | |

^{a}Samples were tested in duplicate in each assay, so 2 titer values are shown for each sample.
Numbers in italics indicate values outside of acceptable range.
GMT-geometric mean titer; HQC-high quality control; ID-identification; LQC-low quality control; MQC-mid quality control; NC-negative control.

TABLE 5
Assay Robustness-upper incubation time limit results

| | | Reference | Upper Incubation | | % | % |
|---|---|---|---|---|---|---|
| Sample | Sample | Condition | Time Limit | | Recov- | Differ- |
| # | ID | Titer | Titera | GMT | ery | ence |
| 1 | HQC | 158.5 | 149.4 | 154 | 97.1 | −2.9 |
| | | | 158.7 | | | |
| 2 | MQC | 66.6 | 64.4 | 62.9 | 94.5 | −5.5 |
| | | | 61.5 | | | |
| 3 | LQC | 37.6 | 39 | 39.3 | 104.5 | 4.5 |
| | | | 39.6 | | | |
| 4 | NC | 5 | 5 | 5 | 100 | 0 |
| | | | 5 | | | |
| 5 | Serum #12 | 11 | 10.3 | 10.8 | 98.5 | −1.5 |
| | | | 11.4 | | | |
| 6 | Serum #13 | 115.5 | 114.4 | 113.5 | 98.3 | −1.7 |
| | | | 112.6 | | | |
| 7 | Serum #14 | 22.3 | 23.8 | 24.4 | 109.6 | 9.6 |
| | | | 25.1 | | | |
| 8 | Serum #15 | 170.3 | 161.7 | 175.6 | 103.1 | 3.1 |
| | | | 190.8 | | | |
| 9 | Serum #16 | 54.7 | 59 | 60.2 | 110.1 | 10.1 |
| | | | 61.5 | | | |
| 10 | Serum #17 | 93.7 | 95.5 | 94.6 | 101 | 1 |
| | | | 93.7 | | | |
| 11 | Serum #18 | 22.4 | 27 | 27.4 | 122.5 | 22.5 |
| | | | 27.9 | | | |
| 12 | Serum #19 | 61.6 | 63.6 | 74 | 120.2 | 20.2 |
| | | | 86.2 | | | |
| 13 | Serum #20 | 18 | 19.1 | 20.1 | 111.8 | 11.8 |
| | | | 21.2 | | | |
| 14 | Serum #21 | 26.9 | 28.7 | 30.1 | 111.8 | 11.8 |
| | | | 31.5 | | | |
| 15 | Serum #22 | 27.9 | 31 | 30.5 | 109.3 | 9.3 |
| | | | 30 | | | |
| 16 | Serum #23 | 62.3 | 61 | 65.5 | 105.2 | 5.2 |
| | | | 70.4 | | | |
| 17 | Serum #24 | 13.6 | 16.3 | 16.1 | 118.4 | 18.4 |
| | | | 15.9 | | | |
| 18 | Serum #1 | 91.4 | 97.6 | 93.6 | 102.4 | 2.4 |
| | | | 89.7 | | | |
| 19 | Serum #2 | 62.4 | 61 | 61.2 | 98.2 | −1.8 |
| | | | 61.5 | | | |
| 20 | Serum #3 | 81.1 | 117.9 | 133.8 | 165 | 65 |
| | | | 151.8 | | | |

^{a}Samples were tested in duplicate in each assay, so 2 titer values are shown for each sample.
Numbers in italics indicate values outside of acceptable range.
GMT-geometric mean titer; HQC-high quality control; ID-identification; LQC-low quality control; MQC-mid quality control; NC-negative control.

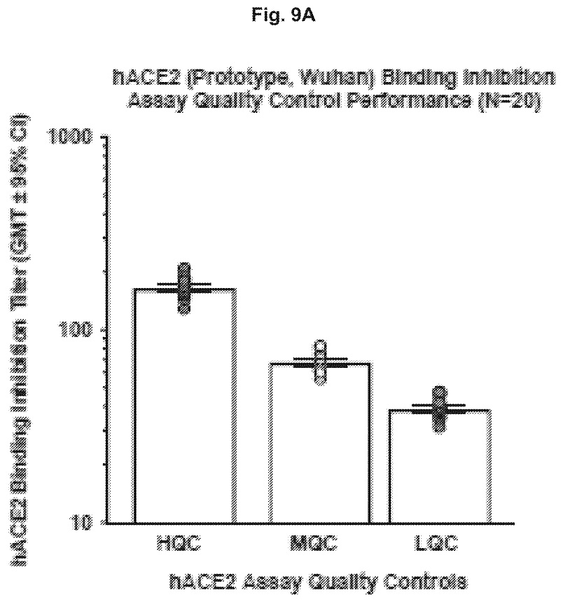

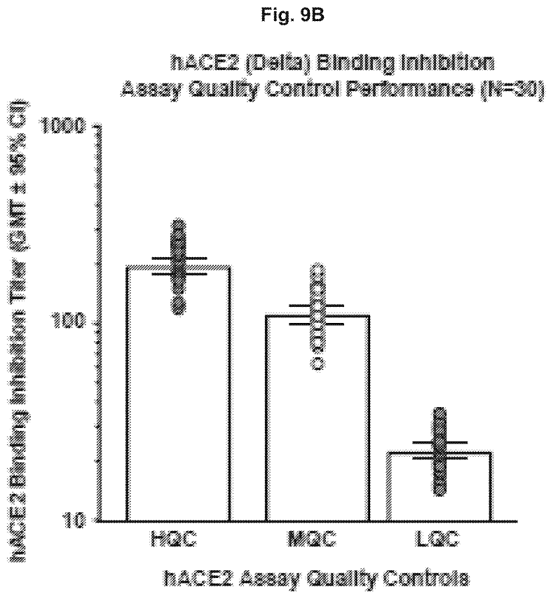

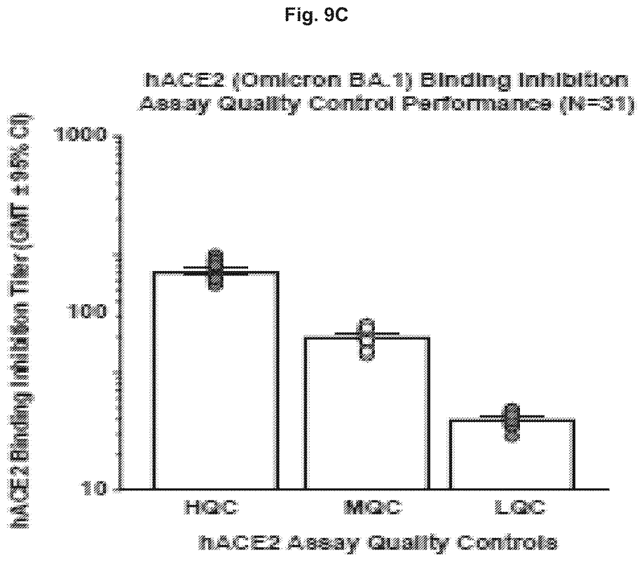

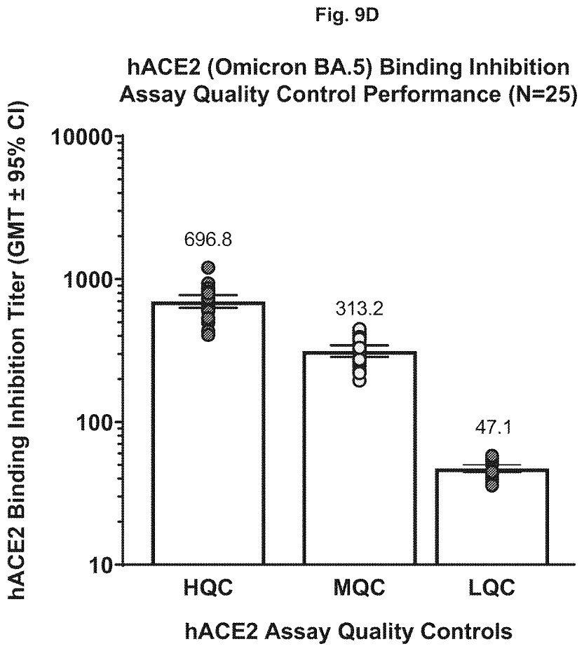

When the serum incubation step was tested at the upper limit of 65 minutes, only 7 of 20 samples (35%) had recovery of 80-120%, but when retested at 62 minutes, 85% of samples were within the 80-120% acceptable range. The original assay was developed using prototype (Wuhan) strain, but controls (different controls for each strain) performed similarly well even when the assay was modified for Delta strain and Omicron BA.1 strain (FIGS. 9A-9C). Similar results for the validation parameters were seen for Delta and Omicron BA.1 strains.

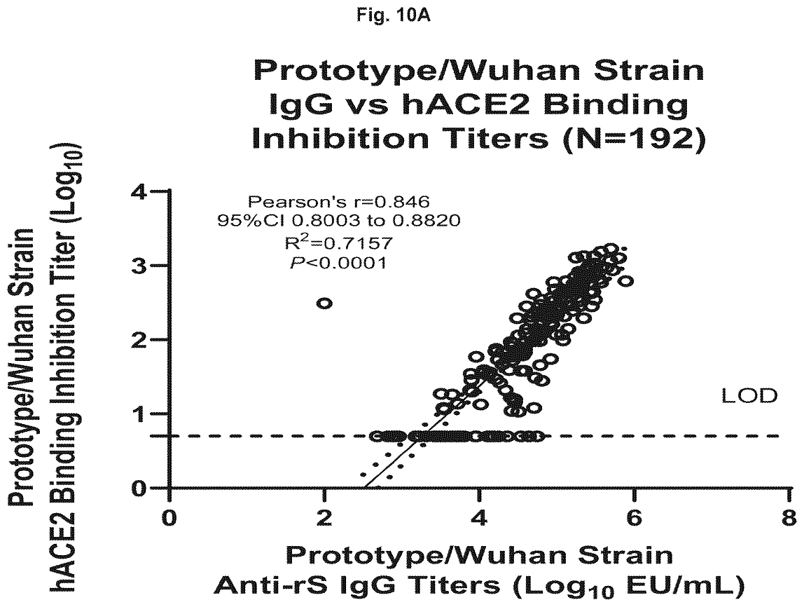

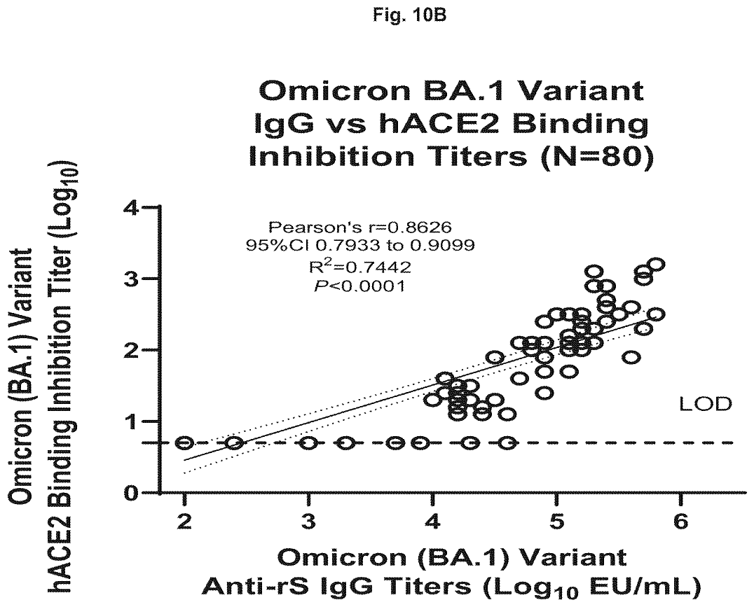

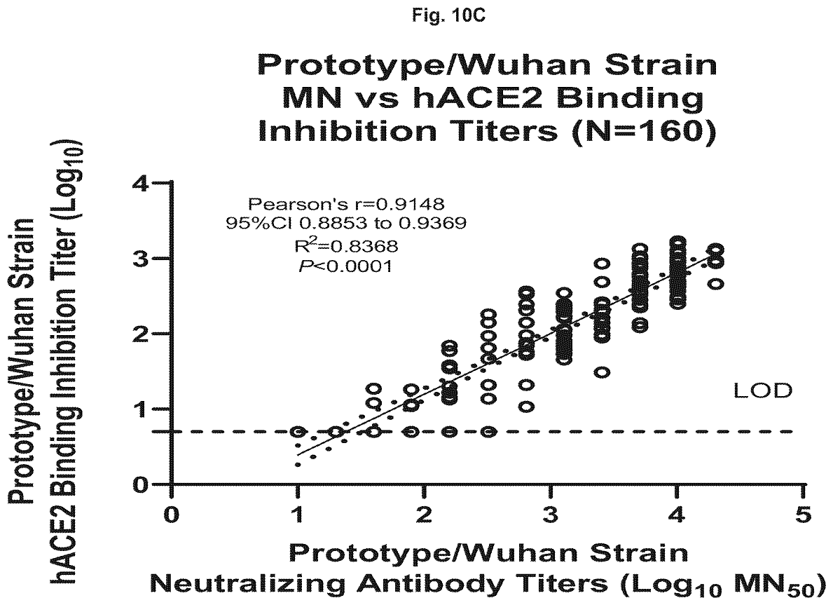

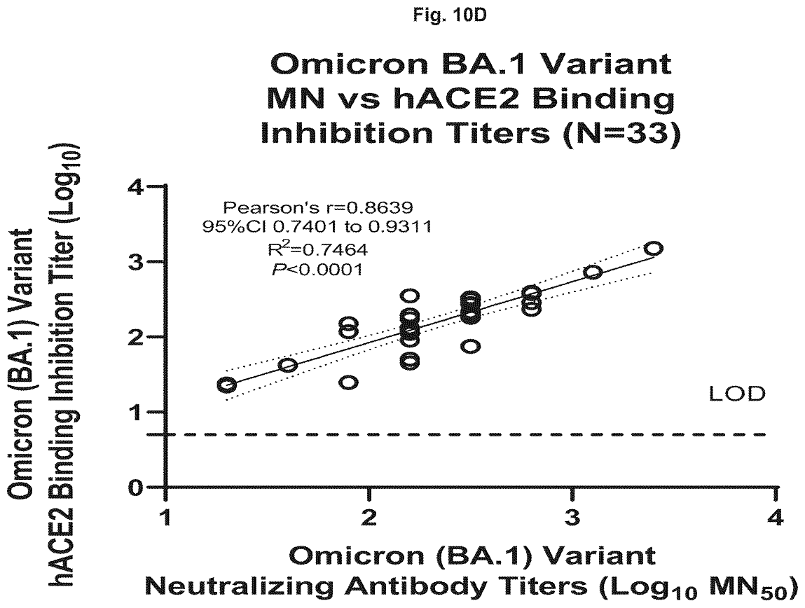

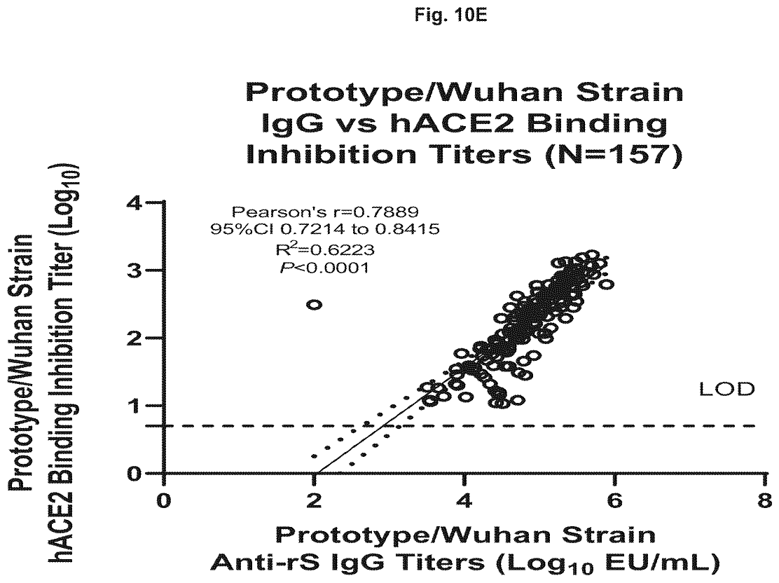

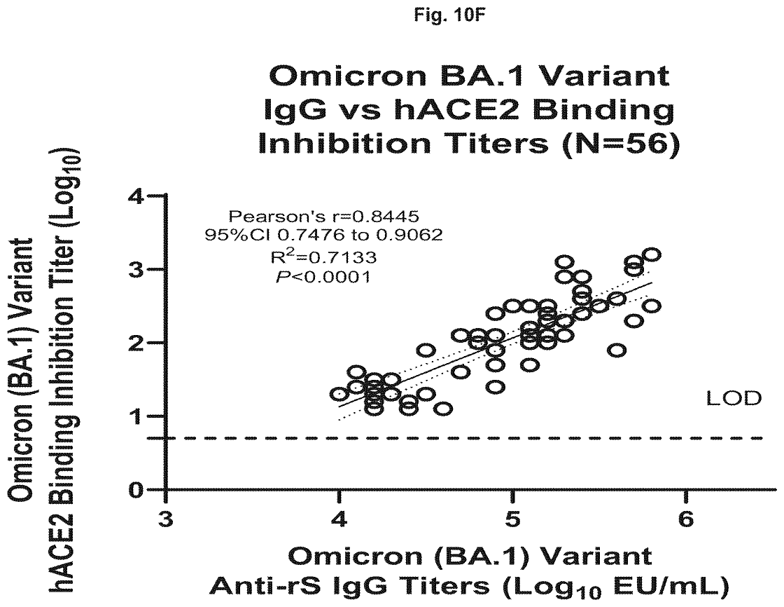

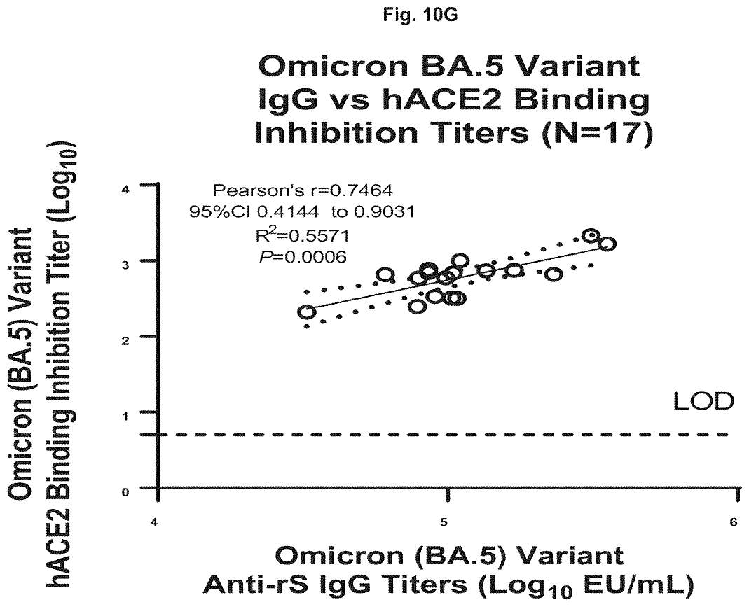

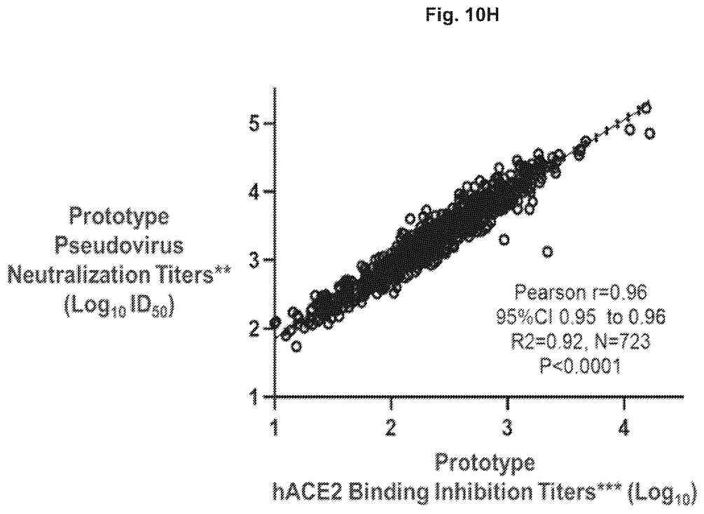

The results from the hACE2 assay were significantly correlated with anti-rS IgG titers for the prototype/Wuhan strain (Pearson's r=0.846, R²=0.7157, P<0.0001) and the Omicron BA.1 variant (Pearson's r=0.8626, R²=0.7442, P<0.0001). Correlations without the data points at LOD values for the hACE2 binding inhibition assay demonstrated similar significant correlations with anti-rS IgG titers for the prototype/Wuhan strain (Pearson's r=0.789, R²=0.6223, P<0.0001) and the Omicron BA.1 variant (Pearson's r=0.8445, R²=0.7113, P<0.0001). hACE2 binding inhibition for Omicron BA.5 variant also showed a similar significant correlation with anti-rS IgG assay data (Pearson's r=0.7464, R²=0.5571, P<0.0006. The results from the hACE2 assay were also significantly correlated with neutralizing antibody titers for the prototype/Wuhan strain (Pearson's r=0.9148, R²=0.8368, P<0.0001) and the Omicron BA.1 variant (Pearson's r=0.8639, R²=0.7464, P<0.0001). The assay performed well for multiple SARS-CoV-2 variants (e.g., Delta and Omicron BA.1/BA.5 variants). (FIGS. 10A-G).

Conclusion: Our results suggest that the ACE2 binding inhibition assay provides a rapid, high-throughput option without biocontainment concerns to evaluate vaccine immunogenicity which correlates with microneutralization and can detect the serologic difference between variants more sensitively than simple anti-spike IgG binding. Along with other clinical biomarkers, ACE2 inhibition assay can provide valuable insights into correlate of protection (CoP) and further enable vaccine development against newly emerging COVID-19 variants.

Additionally, because of the limited throughput and turnaround time of assays associated with biosafety level 3 (BSL-3) procedures, the hACE2 binding inhibition assay conducted in BSL-2 laboratories can add significant value. This procedural difference results in lower assay costs for hACE2 binding assays. As the hACE2 binding inhibition assay is an in vitro assay (without the need for culturing cells) in contrast to the infectious SARS-CoV-2 microneutralization assay, it can also simplify the assay procedure and pro-vide faster assay data on clinical trial samples. Robust correlation of hACE2 binding inhibition with microneutralization assay using infectious SARS-CoV-2 assay (conducted in BSL-3) for both prototype and Omicron BA.1 variant demonstrates the utility of hACE2 binding inhibition as a surrogate for BSL-3-based infectious SARS-CoV-2 neutralization assay.

### NUMBERED EMBODIMENTS OF THE DISCLOSURE

Notwithstanding the appended claims, the disclosure sets forth the following numbered embodiments:
1. A method for measuring the ability of a biological sample to inhibit binding of a SARS-CoV-2 S glycoprotein to human angiotensin-converting enzyme 2 (hACE2), comprising:
   (a) measuring binding of a SARS-CoV-2 S glycoprotein to hACE2 after exposure to a biological sample by:
   (i) coating a surface (e.g., a plate) with the SARS-CoV-2 S glycoprotein;
   (ii) exposing the surface to the biological sample (e.g., human serum);
   (iii) exposing the surface to hACE2; and
   (iv) detecting hACE2 that is bound to the surface;
   (b) measuring binding of a SARS-CoV-2 S glycoprotein to hACE2 in the absence of the biological sample of (a); and
   (c) comparing binding of the SARS-CoV-2 S glycoprotein to hACE2 in (a) and (b); wherein a biological sample inhibits binding of the SARS-CoV-2 S glycoprotein if the amount of bound hACE2 in (a) is less than the amount of bound hACE2 in (b).
2. The method of embodiment 1, wherein the biological sample comprises serum from a patient that was administered a COVID-19 vaccine.
3. The method of embodiment 2, wherein the COVID-19 vaccine comprises (i) a nucleic acid encoding a SARS-CoV-2 S glycoprotein or (ii) a SARS-CoV-2 S glycoprotein.
4. The method of any one of embodiments 1-3, wherein the SARS-CoV-2 S glycoprotein includes the transmembrane domain.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world. The following patent documents are incorporated by reference herein in their entireties: International Publication No. 2021/154812; and International Publication No. 2022/203963; International Publication No. 2023/102448.

1. A method for determining if a biological sample contains antibodies that inhibit binding of a SARS-CoV-2 S glycoprotein to human angiotensin-converting enzyme 2 (hACE2), comprising:
(a) measuring binding of a SARS-CoV-2 S glycoprotein to hACE2 after exposure to a biological sample by:
(i) providing a surface coated with the SARS-CoV-2 S glycoprotein;
(ii) exposing the surface to the biological sample;
(iii) exposing the surface to hACE2; and
(iv) detecting hACE2 that is bound to the surface;
(b) measuring binding of a SARS-CoV-2 S glycoprotein to hACE2 in the absence of the biological sample of (a) by:
(i) providing a surface coated with the SARS-CoV-2 S glycoprotein;
(ii) exposing the surface to hACE2; and
(iii) detecting hACE2 that is bound to the surface; and
(c) comparing binding of the SARS-CoV-2 S glycoprotein to hACE2 in (a) and (b);
wherein a biological sample contains antibodies that inhibit binding of the SARS-CoV-2 S glycoprotein to hACE2 if the amount of bound hACE2 in (a) is less than the amount of bound hACE2 in (b).

2. The method of claim 1, wherein the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of SEQ ID NO: 1, SEQ ID NO: 2, or SEQ ID NO: 3.

3. The method of claim 1, wherein the SARS-CoV-2 S glycoprotein has an inactive furin cleavage site.

4. The method of claim 3, wherein the SARS-CoV-2 S glycoprotein has an inactive furin cleavage site having the amino acid sequence of QQAQ (SEQ ID NO: 68).

5. The method of claim 1, wherein amino acids 973 and 974 of the SARS-CoV-2 S glycoprotein are proline, as compared to a wild-type SARS-CoV-2 S glycoprotein having the amino acid sequence of SEQ ID NO: 2.

6. The method of claim 1, wherein the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 3, 5-13, 15, 17-19, 21, and 23-66.

7. The method of claim 1, wherein the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 3-13, 20, and 22-66.

8. The method of claim 1, wherein the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 3, 5-13, and 23-66.

9. The method of claim 1, wherein the SARS-CoV-2 S glycoprotein is from a SARS-CoV-2 virus or a variant of SARS-CoV-2.

10. The method of claim 9, wherein the variant of SARS-CoV-2 is a B.1.1.7 SARS-CoV-2 strain; a B.1.351 SARS-CoV-2 strain; a P.1 SARS-CoV-2 strain; a Cal.20C SARS-CoV-2 strain; a B.1.617.2 SARS-CoV-2 strain; a B.1.525 SARS-CoV-2 strain; a B.1.526 SARS-CoV-2 strain; a B.1.617.1 SARS-CoV-2 strain; a C.37 SARS-CoV-2 strain; a B.1.621 SARS-CoV-2 strain; or a B.1.1.529 SARS-CoV-2 strain.

11. The method of claim 1, wherein the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a SARS-CoV-2 S glycoprotein from a SARS-CoV-2 S omicron variant selected from the group consisting of: BA.1, BA.2.12.1, BA.2, BA.3, BA.4, BA.5, XBB.1.5, XBB.2.3, and XBB.1.16.

12. The method of claim 1, wherein the hACE2 is attached to a tag.

13. The method of claim 12, wherein the tag is a His tag.

14. The method of claim 1, wherein the biological sample is serum, plasma, blood, saliva, a nasopharyngeal swab, or mucus.

15. The method of claim 1, wherein the biological sample is from a patient who has previously had COVID-19.

16. The method of claim 1, wherein the biological sample is from a patient who has been administered an immunogenic composition against a SARS-CoV-2 virus or a variant thereof.

17. The method of claim 1, wherein the SARS-CoV-2 S glycoprotein includes a transmembrane domain.
